(19) United States
(12) Patent Application Publication
(10) Pub. No.: US 2022/0251141 A1
(43) Pub. Date: Aug. 11, 2022

(54) IMMUNOMODULATORS

(71) Applicant: Bristol-Myers Squibb Company, Princeton, NJ (US)

(72) Inventors: Tao WANG, Farmington, CT (US); Li-Qiang SUN, Glastonbury, CT (US); Zhaoxing MENG, Pennington, NJ (US); Paul SCOLA, Glastonbury, CT (US)

(21) Appl. No.: 17/612,915

(22) PCT Filed: May 21, 2020
(86) PCT No.: PCT/US2020/034053

§ 371 Date: Nov. 19, 2021

Related U.S. Application Data
(60) Provisional application No. 62/850,622, filed on May 21, 2019.

(51) Int. Cl. C07K 7/52

(52) U.S. Cl. CPC C07K 7/52; A61K 38/00

(57) ABSTRACT

In accordance with the present disclosure, macrocyclic compounds have been discovered that bind to PD-L1 and are capable of inhibiting the interaction of PD-L1 with PD-1 and CD80. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This PCT application claims the priority benefit of U.S. Provisional Application No. 62/850,622, filed May 21, 2019, which is incorporated herein by reference in its entirety.

### REFERENCE TO SEQUENCE LISTING SUBMITTED ELECTRONICALLY

The content of the electronically submitted sequence listing in ASCII text file (Name 3338_149PC01_SL_ST25; Size: 5736 bytes; and Date of Creation: May 20, 2020) filed with the application is incorporated herein by reference in its entirety.

### FIELD

The present disclosure provides macrocyclic compounds that bind to PD-L1 and are capable of inhibiting the interaction of PD-L1 with PD-1 and CD80. These macrocyclic compounds exhibit in vitro immunomodulatory efficacy thus making them therapeutic candidates for the treatment of various diseases including cancer and infectious diseases.

### BACKGROUND

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med., 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med., 192:1027-1034 (2000); Latchman et al., Nat. Immunol., 2:261-268 (2001); Carter et al., Eur. J. Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med., 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med., 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99:12293-12297 (2002); Brown et al., J. Immunol., 170:1257-1266 (2003)).

PD-L1 has also been shown to interact with CD80 (Butte M J et al, Immunity; 27:111-122 (2007)). The interaction of PD-L1/CD80 on expressing immune cells has been shown to be an inhibitory one. Blockade of this interaction has been shown to abrogate this inhibitory interaction (Paterson A M, et al., J Immunol., 187:1097-1105 (2011); Yang J, et al. J Immunol. August 1; 187(3):1113-9 (2011)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26: Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

Blockade of PD-1/PD-L1 ligation using antibodies to PD-L1 has been shown to restore and augment T cell activation in many systems. Patients with advanced cancer benefit from therapy with a monoclonal antibody to PD-L1 (Brahmer et al., New Engl. J. Med. (2012)). Preclinical animal models of tumors and chronic infections have shown that blockade of the PD-1/PD-L1 pathway by monoclonal antibodies can enhance an immune response and result in tumor rejection or control of infection. Antitumor immunotherapy via PD-1/PD-L1 blockade can augment therapeutic immune response to a number of histologically distinct tumors (Dong, H. et al., “B7-H1 pathway and its role in the evasion of tumor immunity”, J. Mol. Med., 81(5):281-287 (2003); Dong, H. et al., “Tumor-associated B7-H1 promotes T-cell apoptosis: a potential mechanism of immune evasion”, Nat. Med., 8(8):793-800 (2002)).

Interference with the PD-1/PD-L1 interaction causes enhanced T cell activity in systems with chronic infection. Blockade of PD-L1 caused improved viral clearance and restored immunity in mice with chromoic lymphocytic chorio meningitis virus infection (Barber, D. L. et al., “Restoring function in exhausted CD8 T cells during chronic viral infection”, Nature, 439(7077):682-687 (2006)). Humanized mice infected with HIV-1 show enhanced protection against viremia and viral depletion of CD4+ T cells (Palmer et al., J. Immunol. (2013)). Blockade of PD-1/PD-L1 through monoclonal antibodies to PD-L1 can restore in vitro antigen-specific functionality to T cells from HIV patients (Day, Nature (2006); Petrovas, J. Exp. Med. (2006); Trautman, Nature Med. (2006); D'Souza, J. Immunol. (2007); Zhang, Blood (2007); Kaufmann, Nature Imm. (2007); Kasu, J. Immunol. (2010); Porichis, Blood (2011)), HCV patients (Golden-Mason, J. Virol. (2007); Jeung, J. Leuk. Biol. (2007); Urbani, J. Hepatol. (2008); Nakamoto, PLoS Path. (2009); Nakamoto, Gastroenterology (2008)) and HBV patients (Boni, J. Virol. (2007); Fisicaro, Gastro. (2010); Fisicaro et al., Gastroenterology (2012); Boni et al., Gastro. (2012); Penna et al., J. Hep. (2012); Raziorrough, Hepatology (2009); Liang, World J. Gastro. (2010); Zhang, Gastro. (2008)).

Blockade of the PD-L1/CD80 interaction has also been shown to stimulate immunity (Yang J., et al., J Immunol. August 1; 187(3):1113-9 (2011)). Immune stimulation resulting from blockade of the PD-L1/CD80 interaction has been shown to be enhanced through combination with blockade of further PD-1/PD-L1 or PD-1/PD-L2 interactions.

Alterations in immune cell phenotypes are hypothesized to be an important factor in septic shock (Hotchkiss, et al., Nat Rev Immunol (2013)). These include increased levels of PD-1 and PD-L1 (Guignant, et al, Crit. Care (2011)). Cells from septic shock patients with increased levels of PD-1 and PD-L1 exhibit an increased level of T cell apoptosis. Antibodies directed to PD-L1, can reduce the level of immune cell apoptosis (Zhang et al, Crit. Care (2011)). Furthermore, mice lacking PD-1 expression are more resistant to septic shock symptoms than wildtype mice. Yang J., et al.. J Immunol. August 1; 187(3):1113-9 (2011)). Studies have revealed that blockade of the interactions of PD-L1 using antibodies can suppress inappropriate immune responses and ameliorate disease signs.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med., 205(3):543-555 (2008); Finnefrock, A. C. et al., “PD-1 blockade in rhesus macaques: impact on chronic infection and prophylactic vaccination”, J. Immunol., 182(2):980-987 (2009); Song, M.-Y. et al., “Enhancement of vaccine-induced primary and memory CD8+t-cell responses by soluble PD-1”, J. Immunother., 34(3):297-306 (2011)).

The PD-1 pathway is a key inhibitory molecule in T cell exhaustion that arises from chronic antigen stimulation during chronic infections and tumor disease. Blockade of the PD-1/PD-L1 interaction through targeting the PD-L1 protein has been shown to restore antigen-specific T cell immune functions in vitro and in vivo, including enhanced responses to vaccination in the setting of tumor or chronic infection. Accordingly, agents that block the interaction of PD-L1 with either PD-1 or CD80 are desired.

### SUMMARY

The present disclosure provides macrocyclic compounds which inhibit the PD-1/PD-L1 and CD80/PD-L1 protein/protein interaction, and are thus useful for the amelioration of various diseases, including cancer and infectious diseases.

In a first aspect the present disclosure provides a compound of formula (I)

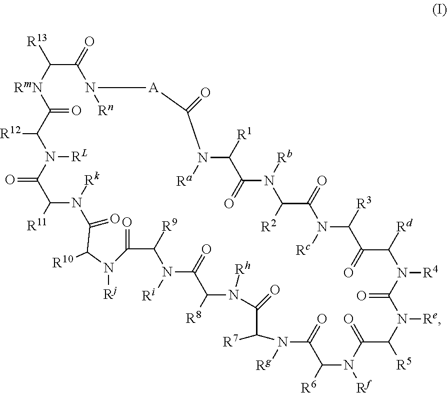

or a pharmaceutically acceptable salt thereof, wherein:

A is selected from a bond,

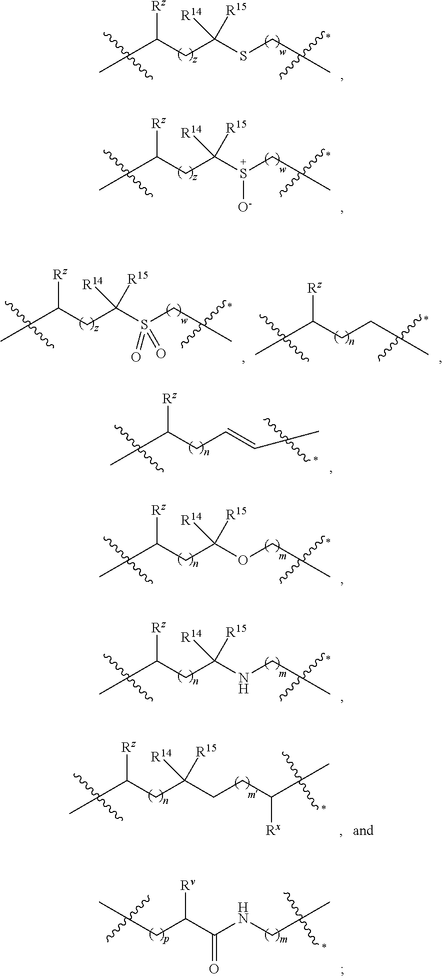

wherein:
[symbol] denotes the point of attachment to the carbonyl group and [symbol] denotes the point of attachment to the nitrogen atom;
z is 0, 1, or 2;
w is 1 or 2;
n is 0 or 1;
m is 1 or 2;
m′ is 0 or 1;
p is 0, 1, or 2;
R^{x} is hydrogen, amino, hydroxy, or methyl;
R¹⁴ and R¹⁵ are independently hydrogen or methyl; and
R^{z} is hydrogen or —C(O)NHR¹⁶; wherein R¹⁶ is hydrogen, —CHR¹⁷C(O)NH₂, —CHR¹⁷C(O)NHCHR¹⁸C(O)NH₂, or —CHR¹⁷C(O)NHCHR¹⁸C(O)NHCH₂C(O)NH₂; wherein R¹⁷ is hydrogen or —CH₂OH and wherein R¹⁸ is hydrogen or methyl;
R^{v} is hydrogen or a natural amino acid side chain;
R^{c}, R^{f}, R^{h}, Rⁱ, and R^{m} are hydrogen;
Rⁿ is hydrogen or methyl or, when p is 0, R^{v} and Rⁿ, together with the atoms to which they are attached, can form a pyrrolidine ring;
R^{a}, R^{e}, and R^{j} are each independently hydrogen or methyl;
R⁵ is —(CH₂)_{q}NR⁵⁰R⁵¹, a natural amino acid side chain, or an unnatural amino acid side chain;
R⁹ is —(CH₂)_{q′}NR^{50′}R^{51′}, a natural amino acid side chain, or an unnatural amino acid side chain;
provided that at least one of R⁵ and R⁹ is other than a natural amino acid side chain or an unnatural amino acid side chain;
q and q′ are each independently 1 or 2;
R⁵⁰, R⁵¹, R^{50′}, and R^{51′} are each independently hydrogen, C₁-C₁₃alkoxycarbonyl, C₄-C₁₃alkylcarbonyl, C₁-C₁₃alkylsulfanylcarbonyl, C₁-C₁₃haloalkoxycarbonyl, C₁-C₁₃haloalkylcarbonyl, —CN, —C(N—CN)C₁-C₁₃alkyl, —C(O)NR⁷⁰R⁷¹, —C(S)NR⁹⁰R91, or —SO₂NR⁹⁰R⁹¹;
R⁷⁰ and R⁷¹ are independently hydrogen, C₁-C₁₃alkoxy, C₁-C₁₃alkyl, C₁-C₁₃alkylcarbonyl, C3-C₁₄cycloalkyl, or phenylC₁-C₃alkyl wherein the phenyl part of the phenylC₁-C₃alkyl is optionally substituted with one, two, or three groups wherein each group is independently C₁-C₃alkoxy, C₁-C₃alkyl, or C₁-C₃alkylcarbonyl, and wherein the phenyl part of the phenylC₁-C₃alkyl is optionally fused to a dioxolanyl ring;
R⁹⁰ and R⁹¹ are independently hydrogen or C₁-C₆alkyl;
provided that when R⁵ is —(CH₂)qNR⁵⁰R⁵¹ and R⁹ is an amino acid side chain or an unnatural amino acid side chain, at least one of R⁵⁰ and R⁵¹ is other than hydrogen;
provided that when R⁹ is —(CH₂)_{q′}NR^{50′}R^{51′} and R⁵ is an amino acid side chain or an unnatural amino acid side chain, at least one of R^{50′} and R^{51′} is other than hydrogen; and
provided that when R⁵ is —(CH₂)_{q′}NR^{50′}R^{51′} and R⁹ is —(CH₂)_{q′}NR^{50′}R^{51′}; at least one of R⁵⁰, R⁵¹, R^{50′} and R^{51′} is other than hydrogen;

R¹, R², R³, R⁴, R⁶, R⁷, R⁸, R¹⁰, R¹¹, R¹², and R¹³ are each independently a natural amino acid side chain or an unnatural amino acid side chain; or

R², R⁴, R⁶, R⁷, R⁸, R¹⁰, R¹¹, R¹², and R¹³ can each independently form a ring with the corresponding vicinal R group as described below;

R^{b} is methyl or R^{b} and R², together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxy;

R^{d} is hydrogen or methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;

R^{g} is hydrogen or methyl, or R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;

R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy; and

R^{L} is methyl or R^{L} and R¹², together with the atoms to which they are attached, form an azetidine or pyrrolidine ring, wherein each ring is optionally substituted with one to four groups wherein each group is independently amino, cyano, methyl, halo, or hydroxy;

provided that the compound of formula (I) contains at least one carbon on the backbone of the ring that has four substituents other than hydrogen and is not an alpha-methyl-substituted ring.

In a first embodiment of the first aspect, the present disclosure provides a compound of formula (I), or a pharmaceutically acceptable salt thereof, wherein A is

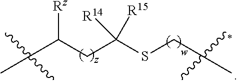

In a second embodiment of the first aspect, z is 0; w is 1; and R^{z} is —C(O)NHR¹⁶. In a third embodiment of the first aspect, R¹⁶ is hydrogen or CHR¹⁷C(O)NH₂, wherein R¹⁷ is hydrogen.

In a fourth embodiment of the first aspect, the present disclosure provides a compound of formula (I), or a pharmaceutically acceptable salt thereof, wherein:

R^{d} is methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;

R^{g} is methyl, or R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group; and

R^{k} is methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy.

In a fifth embodiment of the first aspect, the present disclosure provides a compound of formula (I), or a pharmaceutically acceptable salt thereof, wherein:

R^{d} and R⁴, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;

R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group; and

R^{k} is methyl.

In a sixth embodiment of the first aspect, the present disclosure provides a compound of formula (I), or a pharmaceutically acceptable salt thereof, wherein:

R¹ is biphenylC₁-C₃alkyl wherein the biphenyl is optionally substituted with a methyl group, diphenylmethyl, naphthylC₁-C₃alkyl, phenoxyC₁-C₃alkyl wherein the phenoxy part of the phenoxyC₁-C₃alkyl is optionally substituted with a C₁-C₃alkyl group, or phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, or —OP(O)(OH)₂;

R² is C₁-C₇alkyl, C₂-C₇alkenyl, C₁-C₃alkoxyC₁-C₃alkyl, or C₁-C₃alkylsulfanylC₁-C₃alkyl, or, R² and R^{b}, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxyl;

R³ is C₁-C₆alkoxycarbonylC₁-C₃alkyl, carboxyC₁-C₃alkyl, or NR^{t}R^{u}carbonylC₁-C₃alkyl, wherein R^{t} and R^{u} are independently hydrogen, C₁-C₃alkyl, or triphenylmethyl;

R⁴ and R^{d}, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;

R⁵ is —(CH₂)_{q}NR⁵⁰R⁵¹;

R⁶ is C₁-C₇alkyl, C₂-C₇alkenyl, C₁-C₃alkoxyC₁-C₃alkyl, or C₁-C₃alkylsulfanylC₁-C₃alkyl;

R⁷ and R^{g}, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;

R⁸ and R¹⁰ are each independently azaindolylC₁-C₃alkyl, benzothiazolylC₁-C₃alkyl, benzothienylC₁-C₃alkyl, benzyloxyC₁-C₃alkyl, C₃-C₁₄cycloalkylC₁-C₃alkyl, furanylC₁-C₃alkyl, imidazolylC₁-C₃alkyl, pyridinylC₁-C₃alkyl, thiazolylC₁-C₃alkyl, thienylC₁-C₃alkyl, or indolylC₁-C₃alkyl, wherein the indolyl part of the indolylC₁-C₃alkyl is optionally substituted with one group which is C₁-C₆alkoxycarbonyl, C₁-C₆alkoxycarbonylC₁-C₃alkyl, C₁-C₃alkyl, carboxyC₁-C₃alkyl, halo, haloC₁-C₃alkoxycarbonyl, hydroxy, or phenyl, wherein the phenyl is further optionally substituted by one, two, or three groups wherein each group is independently C₁-C₃alkoxy, C₁-C₃alkyl, or halo;

R⁹ is —(CH₂)_{q′}NR^{50′}R^{51′}; and

R¹¹, R¹², and R¹³ are each independently C₁-C₇alkyl, C₂-C₇alkenyl, C₁-C₃alkoxyC₁-C₃alkyl, or C₁-C₃alkylsulfanylC₁-C₃alkyl.

In a seventh embodiment of the first aspect, the present disclosure provides a compound of formula (I), or a pharmaceutically acceptable salt thereof, wherein:

R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;

R² is C₁-C₇alkyl, or, R² and R^{b}, together with the atoms to which they are attached, form piperidine ring;

R³ is NR^{t}R^{u}carbonylC₁-C₃alkyl, wherein R^{t} and R^{u} are independently hydrogen or C₁-C₃alkyl;

R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁵ is —(CH₂)_{q}NR⁵⁰R⁵¹;

R⁶ is C₁-C₇alkyl;

R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;

R⁹ is —(CH₂)_{q′}NR^{50′}R^{51′}; and

R¹¹, R¹², and R¹³ are each independently C₁-C₇alkyl.

In an eighth embodiment of the first aspect, the present disclosure provides a compound of formula (I), or a pharmaceutically acceptable salt thereof, wherein:

R⁵ is —(CH₂)_{q}NR⁵⁰R⁵¹;

R⁹ is —(CH₂)_{q′}NR^{50′}R^{51′}; and

R⁵⁰, R⁵¹, R^{50′}, and R^{51′} are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹; provided that when R⁵⁰ and R⁵¹ are each hydrogen, at least one of R^{50′} and R^{51′} is other than hydrogen.

In a ninth embodiment of the first aspect, the present disclosure provides a compound of formula (I) wherein:

R⁵ is —(CH₂)_{q}NR⁵⁰R⁵¹;

R⁹ is —(CH₂)_{q′}NR^{50′}R^{51′}; and

R⁵⁰, R⁵¹, R^{50′}, and R^{51′} are each independently hydrogen, C₁-C₁₃alkoxycarbonyl, C₄-C₁₃alkylcarbonyl, or C₁-C₁₃haloalkylcarbonyl; provided that when R⁵⁰ and R⁵¹ are each hydrogen, at least one of R^{50′} and R^{51′} is other than hydrogen.

In a tenth embodiment of the first aspect, the present disclosure provides a compound of formula (I), or a pharmaceutically acceptable salt thereof, wherein:

A is

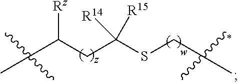

z is 0;

w is 1;

R^{z} is —C(O)NHR¹⁶;

R¹⁶ is hydrogen or CHR¹⁷C(O)NH₂, wherein R¹⁷ is hydrogen;

R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;

R² is C₁-C₇alkyl, or, R² and R^{b}, together with the atoms to which they are attached, form piperidine ring;

R³ is NR^{t}R^{u}carbonylC₁-C₃alkyl, wherein R^{t} and R^{u} are independently hydrogen or C₁-C₃alkyl;

R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁵ is —(CH₂)_{q}NR⁵⁰R⁵¹;

R⁶ is C₁-C₇alkyl;

R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;

R⁹ is —(CH₂)_{q′}NR^{50′}R^{51′}; and

R¹¹, R¹², and R¹³ are each independently C₁-C₇alkyl.

In an eleventh embodiment of the first aspect, the present disclosure provides a compound of formula (I), or a pharmaceutically acceptable salt thereof, wherein:

A is

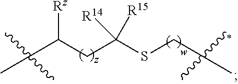

z is 0;

w is 1;

R^{z} is —C(O)NHR¹⁶;

R¹⁶ is hydrogen or CHR¹⁷C(O)NH₂, wherein R¹⁷ is hydrogen;

R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;

R² is C₁-C₇alkyl, or, R² and R^{b}, together with the atoms to which they are attached, form piperidine ring;

R³ is NR^{t}R^{u}carbonylC₁-C₃alkyl, wherein R^{t} and R^{u} are independently hydrogen or C₁-C₃alkyl;

R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁵ is —(CH₂)_{q}NR⁵⁰R⁵¹; wherein R⁵⁰ and R⁵¹ are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹;

R⁶ is C₁-C₇alkyl;

R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;

R⁹ is —(CH₂)_{q′}NR^{50′}R^{51′}; wherein R^{50′} and R^{51′} are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹; provided that when R⁵⁰ and R⁵¹ are each hydrogen, at least one of R^{50′} and R^{51′} is other than hydrogen; and

R¹¹, R¹², and R¹³ are each independently C₁-C₇alkyl.

In a twelfth embodiment of the first aspect, the present disclosure provides a compound of formula (I), or a pharmaceutically acceptable salt thereof, wherein:

A is

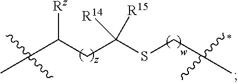

z is 0;

w is 1;

R^{z} is —C(O)NHR¹⁶;

R¹⁶ is CHR¹⁷C(O)NH₂, wherein R¹⁷ is hydrogen;

R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;

R² is C₁-C₇alkyl, or, R² and R^{b}, together with the atoms to which they are attached, form piperidine ring;

R³ is NR^{t}R^{u}carbonylC₁-C₃alkyl, wherein R^{t} and R^{u} are independently hydrogen or C₁-C₃alkyl;

R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁵ is —(CH₂)_{q}NR⁵⁰R⁵¹; wherein R⁵⁰ and R⁵¹ are each independently hydrogen, C₁-C₁₃alkoxycarbonyl, C₄-C₁₃alkylcarbonyl, or C₁-C₁₃haloalkylcarbonyl;

R⁶ is C₁-C₇alkyl;

R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;

R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;

R⁹ is —(CH₂)_{q′}NR^{50′}R^{51′}; wherein R^{50′} and R^{51′} are each independently hydrogen, C₁-C₁₃alkoxycarbonyl, C₄-C₁₃alkylcarbonyl, or C₁-C₁₃haloalkylcarbonyl; provided that when R⁵⁰ and R⁵¹ are each hydrogen, at least one of R^{50′} and R^{51′} is other than hydrogen; and

R¹¹, R¹², and R¹³ are each independently C₁-C₇alkyl.

In a second aspect, the present disclosure provides a compound of formula (II)

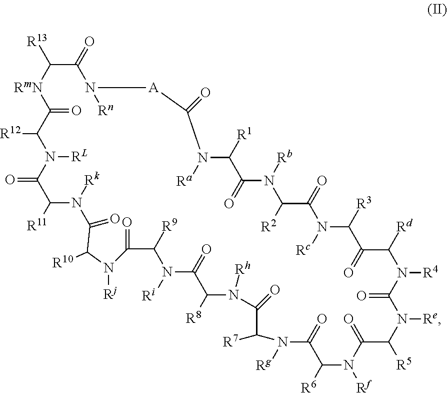

or a pharmaceutically acceptable salt thereof, wherein:

A is selected from a bond,

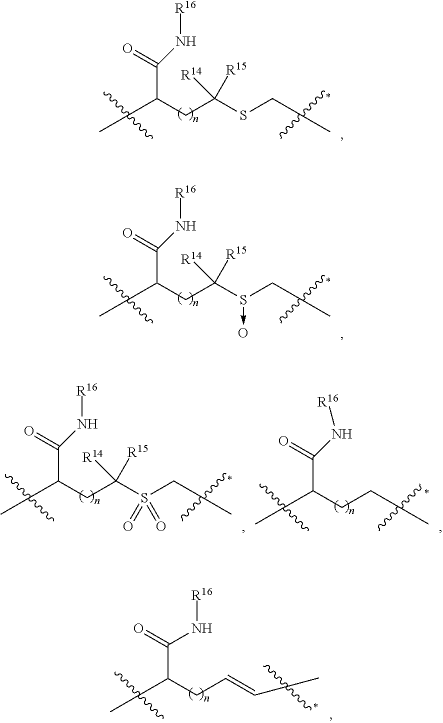

and;
wherein: [symbol]
denotes the point of attachment to the carbonyl group and [symbol] denotes the point of attachment to the nitrogen atom;

n is 0 or 1;

m is 1 or 2;

R¹⁴ and R¹⁵ are independently hydrogen or methyl; and

R¹⁶ is hydrogen, —CHR¹⁷C(O)NH₂, —CHR¹⁷C(O)NHCHR¹⁸C(O)NH₂, or

—CHR¹⁷C(O)NHCHR¹⁸C(O)NHCH₂C(O)NH₂; wherein R′⁷ is hydrogen or —CH₂OH and wherein R¹⁸ is hydrogen or methyl;

R^{f}, R^{h}, Rⁱ, and R^{m} are hydrogen;

Rⁿ is methyl;

R^{a} and R^{j} are each independently hydrogen or methyl;

q and q′ are each independently 1 or 2;

R¹, R², R³, R⁴, R⁵, R⁶, R⁷, R⁸, R⁹, R¹⁰, R¹¹, R¹², and R¹³ are each independently a natural amino acid side chain or an unnatural amino acid side chain; or form a ring with the corresponding vicinal R group as described below;

R^{b} is methyl or R^{b} and R², together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxy;

R^{d} is hydrogen or methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;

R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy;

R^{e} is hydrogen or methyl, or R^{e} and R⁵, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;

R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy;

R^{L} is methyl or R^{L} and R¹², together with the atoms to which they are attached, form an azetidine or pyrrolidine ring, wherein each ring is optionally substituted with one to four groups wherein each group is independently amino, cyano, methyl, halo, or hydroxy;

provided that the compound of formula (I) contains at least one carbon on the backbone of the ring that has four substituents other than hydrogen and is not an alpha-methyl-substituted ring.

In a third aspect, the present disclosure provides a method of enhancing, stimulating, and/or increasing an immune response in a subject in need thereof, wherein the method comprises administering to the subject a therapeutically effective amount of a compound of formula (I) or formula (II), or a pharmaceutically acceptable salt thereof.

In a fourth aspect, the present disclosure provides a method of blocking the interaction of PD-L1 with PD-1 and/or CD80 in a subject, wherein the method comprises administering to the subject a therapeutically effective amount of a compound of formula (I) or formula (II) or a pharmaceutically acceptable salt thereof.

In a fifth aspect the present disclosure provides a method of enhancing, stimulating, and/or increasing an immune response in a subject in need thereof, said method comprising administering to the subject a therapeutically effective amount of a compound of formula (I) or formula (II), or a pharmaceutically acceptable salt thereof. In a first embodiment of the second aspect the method further comprises administering an additional agent prior to, after, or simultaneously with the compound of formula (I), compound of formula (I)), or a pharmaceutically acceptable salt thereof. In a second embodiment the additional agent is selected from an antimicrobial agent, an antiviral agent, a cytotoxic agent, a TLR7 agonist, a TLR8 agonist, an HDAC inhibitor, and an immune response modifier.

In a sixth aspect the present disclosure provides a method of inhibiting growth, proliferation, or metastasis of cancer cells in a subject in need thereof, said method comprising administering to the subject a therapeutically effective amount a compound of formula (I) or formula (II), or a pharmaceutically acceptable salt thereof. In a first embodiment of the third aspect the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In a seventh aspect the present disclosure provides a method of treating an infectious disease in a subject in need thereof, the method comprising administering to the subject a therapeutically effective amount of a compound of formula (I) or formula (II), or a pharmaceutically acceptable salt thereof. In a first embodiment of the fourth aspect the infectious disease is caused by a virus. In a second embodiment the virus is selected from HIV, Hepatitis A, Hepatitis B, Hepatitis C, herpes viruses, and influenza.

In an eighth aspect the present disclosure provides a method of treating septic shock in a subject in need thereof, the method comprising administering to the subject a therapeutically effective amount of a compound of formula (I) or formula (II), or a pharmaceutically acceptable salt thereof.

In a ninth aspect the present disclosure provides a method of blocking the interaction of PD-L1 with PD-1 and/or CD80 in a subject, said method comprising administering to the subject a therapeutically effective amount of a compound of formula (I) or formula (II), or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The terms “natural amino acid side chain” and “naturally occurring amino acid side chain”, as used herein, refer to side chain of any of the naturally occurring amino acids (i.e., alanine, arginine, asparagine, aspartic acid, cysteine, glutamine, glutamic acid, glycine, -histidine, isoleucine, leucine, lysine, methionine, phenylalanine, proline, serine, threonine, tryptophan, tyrosine, and valine) usually in the S-configuration (i.e., the L-amino acid).

The terms “unnatural amino acid side chain” and “non-naturally occurring amino acid side chain”, as used herein, refer to a side chain of any naturally occurring amino acid usually in the R-configuration (i.e., the D-amino acid) or to a group other than a naturally occurring amino acid side chain in R- or S-configuration (i.e., the D- or L-amino acid, respectively) selected from:

C₂-C₇alkenyl, C₁-C₃alkoxyC₁-C₃alkyl, C₁-C₆alkoxycarbonylC₁-C₃alkyl, C₁-C₇alkyl, C₁-C₃alkylsulfanylC₁-C₃alkyl, amidoC₁-C₃alkyl, aminoC₁-C₃alkyl, azaindolylC₁-C₃alkyl, benzothiazolylC₁-C₃alkyl, benzothienylC₁-C₃alkyl, benzyloxyC₁-C₃alkyl, carboxyC₁-C₃alkyl, C₃-C₁₄cycloalkylC₁-C₃alkyl, diphenylmethyl, furanylC₁-C₃alkyl, imidazolylC₁-C₃alkyl, naphthylC₁-C₃alkyl, pyridinylC₁-C₃alkyl, thiazolylC₁-C₃alkyl, thienylC₁-C₃alkyl;

biphenylC₁-C₃alkyl wherein the biphenyl is optionally substituted with a methyl group;

heterocyclyl optionally substituted with one, two, three, four, or five groups independently selected from C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂;

indolylC₁-C₃alkyl, wherein the indolyl part is optionally substituted with one group selected from C₁-C₃alkoxycarbonylC₁-C₃alkyl, C₁-C₃alkyl, carboxyC₁-C₃alkyl, halo, hydroxy, and phenyl, wherein the phenyl is further optionally substituted by one, two, or three groups independently selected from C₁-C₃alkoxy, C₁-C₃alkyl, and halo;

NR^{x}R^{y}(C₁-C₇alkyl), wherein R^{x} and R^{y} are independently selected from hydrogen, C₂-C₄alkenyloxycarbonyl, C₁-C₃alkyl, C₁-C₃alkylcarbonyl, C₃-C₁₄cycloalkylcarbonyl, furanylcarbonyl, and phenylcarbonyl. When the alkyl linker contains more than one carbon, an additional NR^{x}R^{y} group can be on the chain.

NR^{t}R^{u}carbonylC₁-C₃alkyl, wherein R^{t} and R^{u} are independently selected from hydrogen, C₁-C₃alkyl, and triphenylmethyl;

phenyl optionally substituted with one, two, three, four, or five groups independently selected from C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂;

phenylC₁-C₃alkyl wherein the phenyl part is optionally substituted with one, two, three, four, or five groups independently selected from C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂; and

phenoxyC₁-C₃alkyl wherein the phenyl is optionally substituted with a C₁-C₃alkyl group.

The term “C₂-C₄alkenyl”, as used herein, refers to a straight or branched chain group of two to four carbon atoms containing at least one carbon-carbon double bond.

The term “C₂-C₇alkenyl”, as used herein, refers to a straight or branched chain group of two to seven carbon atoms containing at least one carbon-carbon double bond.

The term “C₂-C₄alkenyloxy”, as used herein, refers to a C₂-C₄alkenyl group attached to the parent molecular moiety through an oxygen atom.

The term “C₂-C₄alkenyloxycarbonyl”, as used herein, refers to a C₂-C₄alkenyloxy group attached to the parent molecular moiety through a carbonyl group.

The term “C₁-C₃alkoxy”, as used herein, refers to a C₁-C₃alkyl group attached to the parent molecular moiety through an oxygen atom.

The term “C₁-C₄alkoxy”, as used herein, refers to a C₁-C₄alkyl group attached to the parent molecular moiety through an oxygen atom.

The term “C₁-C₆alkoxy”, as used herein, refers to a C₁-C₆alkyl group attached to the parent molecular moiety through an oxygen atom.

The term “C₁-C₁₃alkoxy”, as used herein, refers to a C₁-C₁₃alkyl group attached to the parent molecular moiety through an oxygen atom.

The term “C₁-C₃alkoxyC₁-C₃alkyl”, as used herein, refers to a C₁-C₃alkoxy group attached to the parent molecular moiety through a C₁-C₃alkyl group.

The term “C₁-C₆alkoxycarbonyl”, as used herein, refers to a C₁-C₆alkoxy group attached to the parent molecular moiety through a carbonyl group.

The term “C₁-C₃alkoxycarbonylC₁-C₃alkyl”, as used herein, refers to a C₁-C₃alkoxycarbonyl group attached to the parent molecular moiety through a C₁-C₃alkyl group.

The term “C₁-C₆alkoxycarbonylC₁-C₃alkyl”, as used herein, refers to a C₁-C₆alkoxycarbonyl group attached to the parent molecular moiety through a C₁-C₃alkyl group.

The term “C₁-C₃alkyl”, as used herein, refers to a group derived from a straight or branched chain saturated hydrocarbon containing from one to three carbon atoms.

The term “C₁-C₄alkyl”, as used herein, refers to a group derived from a straight or branched chain saturated hydrocarbon containing from one to four carbon atoms.

The term “C₁-C₆alkyl”, as used herein, refers to a group derived from a straight or branched chain saturated hydrocarbon containing from one to six carbon atoms.

The term “C₁-C₇alkyl”, as used herein, refers to a group derived from a straight or branched chain saturated hydrocarbon containing from one to seven carbon atoms.

The term “C₁-C₁₃alkyl”, as used herein, refers to a group derived from a straight or branched chain saturated hydrocarbon containing from one to thirteen carbon atoms.

The term “C₄-C₁₃alkyl”, as used herein, refers to a group derived from a straight or branched chain saturated hydrocarbon containing from four to thirteen carbon atoms.

The term “C₁-C₃alkylcarbonyl”, as used herein, refers to a C₁-C₃alkyl group attached to the parent molecular moiety through a carbonyl group.

The term “C₁-C₁₃alkylcarbonyl”, as used herein, refers to a C₁-C₁₃alkyl group attached to the parent molecular moiety through a carbonyl group.

The term “C₄-C₁₃alkylcarbonyl”, as used herein, refers to a C₄-C₁₃alkyl group attached to the parent molecular moiety through a carbonyl group.

The term “C₁-C₃alkylsulfanyl”, as used herein, refers to a C₁-C₃alkyl group attached to the parent molecular moiety through a sulfur atom.

The term “C₁-C₁₃alkylsulfanyl”, as used herein, refers to a C₁-C₁₃alkyl group attached to the parent molecular moiety through a sulfur atom.

The term “C₁-C₃alkylsulfanylC₁-C₃alkyl”, as used herein, refers to a C₁-C₃alkylsulfanyl group attached to the parent molecular moiety through a C₁-C₃alkyl group.

The term “C₁-C₁₃alkylsulfanylcarbonyl”, as used herein, refers to a C₁-C₁₃alkylsulfanyl group attached to the parent molecular moiety through a carbonyl group.

The term “C₁-C₃alkylsulfonyl”, as used herein, refers to a C₁-C₃alkyl group attached to the parent molecular moiety through a sulfonyl group.

The term “C₁-C₃alkylsulfonylamino”, as used herein, refers to a C₁-C₃alkylsulfonyl group attached to the parent molecular moiety through an amino group.

The term “amido”, as used herein, refers to —C(O)NH₂.

The term “amidoC₁-C₃alkyl”, as used herein, refers to an amido group attached to the parent molecular moiety through a C₁-C₃alkyl group.

The term “amino”, as used herein, refers to —NH₂.

The term “aminoC₁-C₃alkyl”, as used herein, refers to an amino group attached to the parent molecular moiety through a C₁-C₃alkyl group.

The term “aminosulfonyl”, as used herein, refers to an amino group attached to the parent molecular moiety through a sulfonyl group.

The term “azaindolylC₁-C₃alkyl”, as used herein, refers to an azaindolyl group attached to the parent molecular through a C₁-C₃alkyl group. The azaindolyl group can be attached to the alkyl moiety through any substitutable atom in the group.

The term “benzothiazolylC₁-C₃alkyl”, as used herein, refers to an benzothiazolyl group attached to the parent molecular through a C₁-C₃alkyl group. The benzothiazolyl group can be attached to the alkyl moiety through any substitutable atom in the group.

The term “benzothienylC₁-C₃alkyl”, as used herein, refers to a benzothienyl group attached to the parent molecular through a C₁-C₃alkyl group. The benzothienyl group can be attached to the alkyl moiety through any substitutable atom in the group.

The term “benzyl”, as used herein, refers to a phenyl group attached to the parent molecular moiety through a CH₂ group.

The term “benzyloxy”, as used herein, refers to a benzyl group attached to the parent molecular moiety through an oxygen atom.

The term “benzyloxyC₁-C₃alkyl”, as used herein, refers to a benzyloxy group attached to the parent molecular moiety through a C₁-C₃alkyl group.

The term “biphenylC₁-C₃alkyl”, as used herein, refers to a biphenyl group attached to the parent molecular moiety through a C₁-C₃alkyl group. The biphenyl group can be attached to the alkyl moiety through any substitutable atom in the group.

The term “carbonyl”, as used herein, refers to —C(O)—.

The term “carboxy”, as used herein, refers to —CO₂H.

The term “carboxyC₁-C₃alkyl”, as used herein, refers to a carboxy group attached to the parent molecular moiety through a C₁-C₃alkyl group.

The term “cyano”, as used herein, refers to —CN.

The term “C₃-C₁₄cycloalkyl”, as used herein, refers to a saturated monocyclic or bicyclic hydrocarbon ring system having three to fourteen carbon atoms and zero heteroatoms. The bicyclic rings can be fused, spirocyclic, or bridged. Representative examples of cycloalkyl groups include, but are not limited to, cyclopropyl, cyclopentyl, octahydropentalene, and bicyclo[3.1.1]heptyl.

The term “C₃-C₁₄cycloalkylC₁-C₃alkyl”, as used herein, refers to a C₃-C₁₄cycloalkyl group attached to the parent molecular moiety through a C₁-C₃alkyl group.

The term “C₃-C₁₄cycloalkylcarbonyl”, as used herein, refers to a C₃-C₁₄ cycloalkyl group attached to the parent molecular moiety through a carbonyl group.

The term “diphenylmethyl”, as used herein, refers to (Ph)₂CH—, wherein each Ph is a phenyl ring.

The term “furanylC₁-C₃alkyl”, as used herein, refers to a furanyl group attached to the parent molecular moiety through a C₁-C₃alkyl group. The furanyl group can be attached to the alkyl moiety through any substitutable atom in the group.

The term “furanylcarbonyl”, as used herein, refers to a furanyl group attached to the parent molecular moiety through a carbonyl group.

The terms “halo” and “halogen”, as used herein, refer to F, Cl, Br, or I.

The term “haloC₁-C₁₃alkoxy”, as used herein, refers to a haloC₁-C₁₃alkyl group attached to the parent molecular moiety through an oxygen atom

The term “haloC₁-C₁₃alkoxycarbonyl”, as used herein, refers to a haloC₁-C₁₃alkoxy group attached to the parent molecular moiety through a carbonyl group.

The term “haloC₁-C₃alkyl”, as used herein, refers to a C₁-C₃alkyl group substituted with one, two, or three halogen atoms.

The term “haloC₁-C₁₃alkyl”, as used herein, refers to a C₁-C₁₃alkyl group substituted with one, two, three, four, five, six, seven, eight, or nine halogen atoms.

The term “haloC₁-C₁₃alkylcarbonyl”, as used herein, refers to a haloC₁-C₁₃alkyl attached to the parent molecular moiety through a carbonyl group.

The term “heterocyclyl”, as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure are attached to the parent molecular moiety through a carbon atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “hydroxy”, as used herein, refers to —OH.

The term “imidazolylC₁-C₃alkyl”, as used herein, refers to an imidazolyl group attached to the parent molecular moiety through a C₁-C₃alkyl group. The imidazolyl group can be attached to the alkyl moiety through any substitutable atom in the group.

The term “indolylC₁-C₃alkyl”, as used herein, refers to an indolyl group attached to the parent molecular moiety through a C₁-C₃alkyl group. The indolyl group can be attached to the alkyl moiety through any substitutable atom in the group.

The term “isoquinolinyloxy”, as used herein, refers to an isoquinoline group attached to the parent molecular moiety through an oxygen atom. The isoquinoline group can be attached to the oxygen atom through any substitutable carbon atom in the group.

The term “naphthylC₁-C₃alkyl”, as used herein, refers to a naphthyl group attached to the parent molecular moiety through a C₁-C₃alkyl group. The naphthyl group can be attached to the alkyl moiety through any substitutable atom in the group.

The term “nitro”, as used herein, refers to —NO₂.

The term “NR^{x}R^{y}”, as used herein, refers to two groups, R^{x} and R^{y}, which are attached to the parent molecular moiety through a nitrogen atom. R^{x} and R^{y} are independently selected from hydrogen, C₂-C₄alkenyloxycarbonyl, C₁-C₃alkylcarbonyl, C₃-C₁₄cycloalkylcarbonyl, furanylcarbonyl, and phenylcarbonyl.

The term “NR^{x}R^{y}(C₁-C₇)alkyl”, as used herein, refers to an NR^{x}R^{y} group attached to the parent molecular moiety through a C₁-C₇alkyl group.

The term “NR^{t}R^{u}”, as used herein, refers to two groups, R^{t} and R^{u}, which are attached to the parent molecular moiety through a nitrogen atom. R^{t} and R^{u} are independently selected from hydrogen, C₁-C₃alkyl, and triphenylmethyl.

The term “NR^{t}R^{u}carbonyl”, as used herein, refers to an NR^{t}R^{u} group attached to the parent molecular moiety through a carbonyl group.

The term “NR^{t}R^{u}carbonylC₁-C₃alkyl”, as used herein, refers to an NR^{t}R^{u}carbonyl group attached to the parent molecular moiety through a C₁-C₃alkyl group.

The tem “phenoxy”, as used herein, refers to a phenyl group attached to the parent molecular moiety through an oxygen atom.

The term “phenoxyC₁-C₃alkyl”, as used herein, refers to a phenoxy group attached to the parent molecular moiety through a C₁-C₃alkyl group.

The term “phenylC₁-C₃alkyl”, as used herein, refers to a phenyl group attached to the parent molecular moiety through a C₁-C₃alkyl group.

The term “phenylcarbonyl”, as used herein, refers to a phenyl group attached to the parent molecular moiety through a carbonyl group.

The term “pyridinylC₁-C₃alkyl”, as used herein, refers to a pyridinyl group attached to the parent molecular moiety through a C₁-C₃alkyl group. The pyridinyl group can be attached to the alkyl moiety through any substitutable atom in the group.

The term “quinolinyloxy”, as used herein, refers to a quinoline group attached to the parent molecular moiety through an oxygen atom. The quinoline group can be attached to the oxygen atom through any substitutable carbon atom in the group.

The term “sulfanyl”, as used herein, refers to —S—.

The term “sulfonyl”, as used herein, refers to —SO₂—.

The term “thiazolylC₁-C₃alkyl”, as used herein, refers to a thiazolyl group attached to the parent molecular moiety through a C₁-C₃alkyl group. The thiazolyl group can be attached to the alkyl moiety through any substitutable atom in the group.

The term “thienylC₁-C₃alkyl”, as used herein, refers to a thienyl group attached to the parent molecular moiety through a C₁-C₃alkyl group. The thienyl group can be attached to the alkyl moiety through any substitutable atom in the group.

The term “triphenylmethyl”, as used herein, refers to —C(Ph)₃, wherein each Ph is a phenyl group.

An “adverse event” or “AE” as used herein is any unfavorable and generally unintended, even undesirable, sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, and benign prostatic hypertrophy).

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-LI”, and “B7-H1” are used interchangeably, and include variants, isoforms, species homologs of human PD-L1, and analogs having at least one common epitope with PD-L1. The complete PD-L1 sequence can be found under GENBANK® Accession No. NP 054862.

The terms “Programmed Death 1”, “Programmed Cell Death 1”, “Protein PD-1”, “PD-1”, “PD1”, “hPD-1” and “hPD-I” are used interchangeably, and include variants, isoforms, species homologs of human PD-1, and analogs having at least one common epitope with PD-1. The complete PD-1 sequence can be found under GENBANK® Accession No. U64863.

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritaration using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

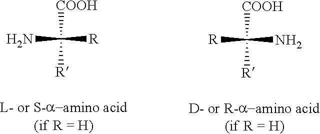

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R”′(prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1 and/or CD80 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration. Thus, it is not useful to specify an exact effective amount in advance.

In another aspect, the disclosure pertains to methods of inhibiting growth of tumor cells in a subject using the macrocyclic compounds of the present disclosure. As demonstrated herein, the compounds of the present disclosure are capable of binding to PD-L1, disrupting the interaction between PD-L1 and PD-1, competing with the binding of PD-L1 with anti-PD-1 monoclonal antibodies that are known to block the interaction with PD-1, enhancing CMV-specific T cell IFNγ secretion, and enhancing HIV-specific T cell IFNγ secretion. As a result, the compounds of the present disclosure are useful for modifying an immune response, treating diseases such as cancer or infectious disease, stimulating a protective autoimmune response or to stimulate antigen-specific immune responses (e.g., by co-administration of PD-L1 blocking compounds with an antigen of interest).

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least none emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and examples are defined as follows: Et₃N or TEA for trimethylamine; iPrNEt₂ or DIPEA or DIEA for diisopropylethylamine; THF for tetrahydrofuran; DME for 1,2-dimethoxyethane; MeOH for methanol; EtOH for ethanol; HCTU for 1-[bis(dimethylamino)methylene]-5-chlorobenzotriazolium 3-oxide hexafluorophosphate or N,N,N′,N′-tetramethyl-O-(6-chloro-1H-benzotriazol-1-yl)uronium hexafluorophosphate; HATU for 1-[bis(dimethylamino)methylene]-1H-1,2,3-triazolo[4,5-b]pyridinium 3-oxide hexafluorophosphate or N-[(dimethylamino)-1H-1,2,3-triazolo-[4,5-b]pyridin-1-ylmethylene]-N-methylmethanaminium hexafluorophosphate N-oxide; HOBt for 1-hydroxybenzotriazole hydrate; DMF for N,N-dimethylformamide; min or mins for minutes; h or hr or hrs for hours; ACN or MeCN for acetonitrile; rt″ for room temperature or retention time (context will dictate); TFA for trifluoroacetic acid; EtOAc for ethyl acetate; and DTT for dithiothreitol (Cleland's reagent).

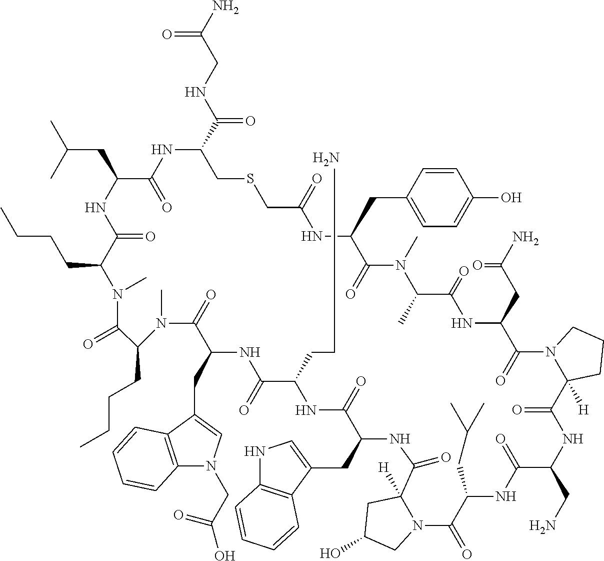 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

### Example 1

Example 1 was prepared according to the procedure described in WO2014/151634.

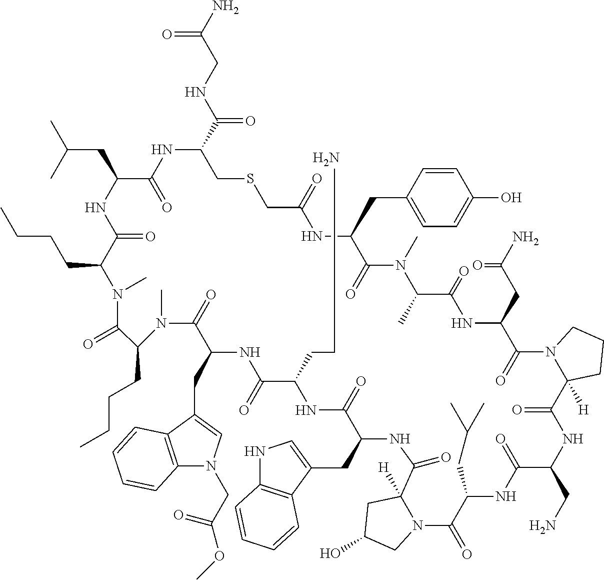 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)OC)c3ccccc23)C(=O)N1C]

### Example 2

(Diazomethyl)trimethylsilane (0.079 mL, 2M in ether) was added into a solution of Example 1 (100 mg) in 2 mL of THF/MeOH (4/1). The reaction was stirred at room temperature for 24 hours. After all the solvents were removed under vacuum, the residue was purified by the preparative HPLC to provide the desired product.

### General Procedure for Compound Preparation:

A mixture of Example 1 or Example 2 (1 eq.), the appropriate electrophile (1-20 eq.) and Et₃N or iPr₂NEt (0-200 eq.) in THF, dioxane, DME, MeOH, or EtOH was stirred at room temperature to 100° C. for 0.5 to 48 hours, then quenched with methanol or water. After the solvents were removed under vacuum, the residue was purified by the preparative HPLC to give the compound.

### Alternative Procedure I for Compound Preparation

Et₃N or iPr₂NEt (1-200 eq.) was added into a solution of the appropriate electrophile (1-20 eq.), HCTU, HATU, or HOBt (1-20 eq.) in DMF, THF, dioxane, or DME. After the mixture was stirred at room temperature for 24 h, Example 1 or 2 (1 eq.) was added. The reaction was then stirred at room temperature to 100° C. for 0.5 to 48 hours, then quenched with methanol or water. After the solvents were removed under vacuum, the residue was purified by the preparative HPLC to give the compound.

### Alternative Procedure II for Compound Preparation

A mixture of Example 1 or Example 2 (1 eq.), the first electrophile (1-20 eq.), and Et₃N or iPr₂NEt (0-200 eq.) in THF, dioxane, DME, MeOH, or EtOH was stirred at room temperature to 100° C. for 0.5 to 48 hours. Then, the second electrophile (1-20 eq.) was added and the resulting mixture was stirred at room temperature to 100° C. for 0.5 to 48 hours, then quenched with methanol or water. After the solvents were removed under vacuum, the residue was purified by preparative HPLC to give the compound.

The compounds shown in Table 1 were prepared from Example 1 or Example 2 using the procedures described above.

TABLE 1
Compound 1003

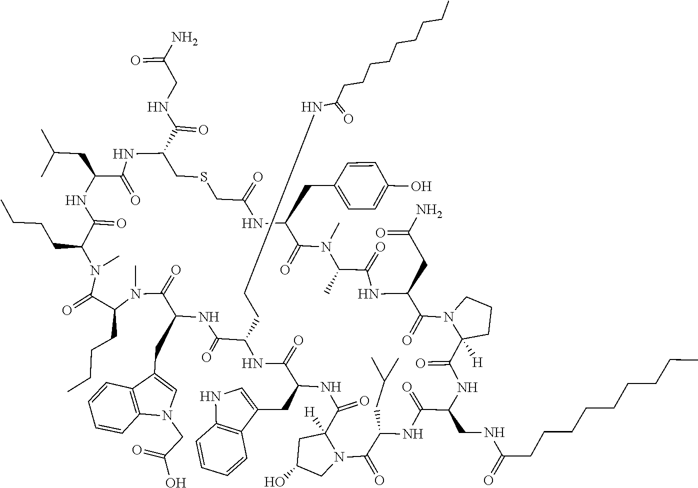

Agents Used

| | |
|---|---|
| Starting Material | Example 1 |
| Electrophile | Decanoyl Chloride |

MS

| | |
|---|---|
| MS (M/2 + H)⁺ Calcd. | 1098 |
| MS (M/2 + H)⁺ Observ. | 1098 |
| Retention Time | 1.39 min |

LC Condition

| | |
|---|---|
| Solvent A | 5% ACN: 95% Water: 10 mM Ammonium Acetate |
| Solvent B | 95% ACN: 5% Water: 10 mM Ammonium Acetate |
| Start % B | 20 |
| Final % B | 100 |
| Gradient Time | 1.5 min |
| Flow Rate | 1 mL/min |
| Wavelength | 220 |
| Temperature | 40° C. |
| Column | Phenomenex LUNA C18, 30 × 2, 3 μ |

Compound 1004

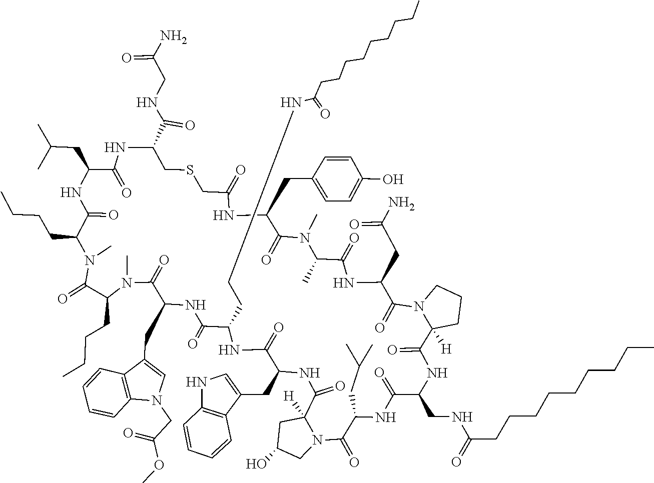

Agents Used

| | |
|---|---|
| Starting Material | Example 1 |
| Electrophile | Decanoyl Chloride |

MS

| | |
|---|---|
| MS (M/2 + H)⁺ Calcd. | 1105 |
| MS (M/2 + H)⁺ Observ. | 1105 |
| Retention Time | 2.69 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1005

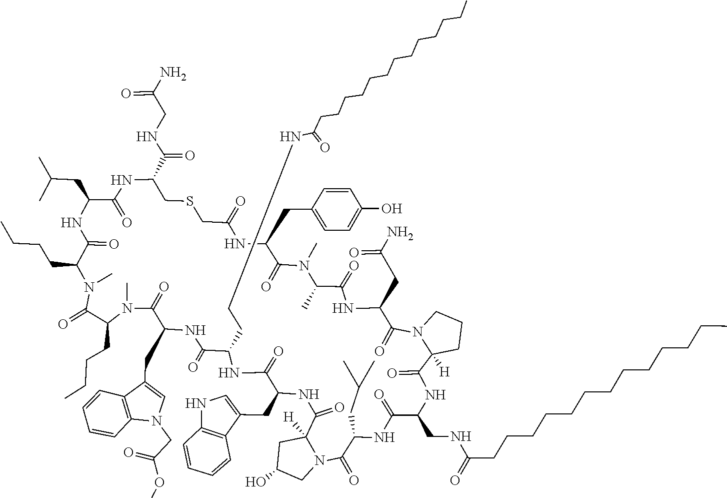

Agents Used

| | |
|---|---|
| Starting Material | Example 1 |
| Electrophile | Tetradecanoyl Chloride |

MS

| | |
|---|---|
| MS (M/2 + H)⁺ Calcd. | 1161 |
| MS (M/2 + H)⁺ Observ. | 1161 |
| Retention Time | 3.26 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1006

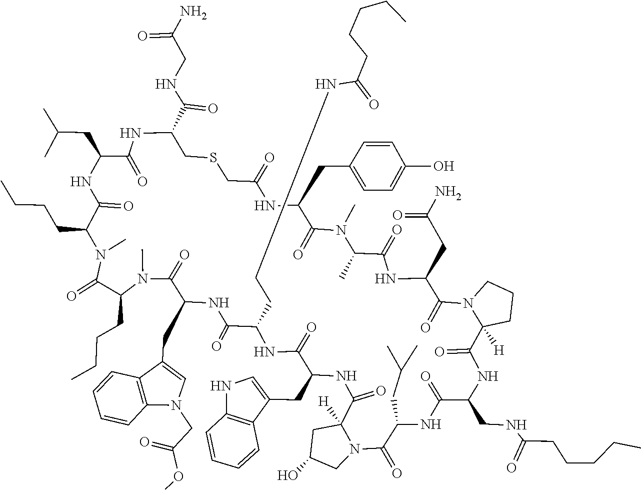

Agents Used

| | |
|---|---|
| Starting Material | Example 2 |
| Electrophile | Hexanoyl Chloride |

MS

| | |
|---|---|
| MS (M/2 + H)⁺ Calcd. | 1049 |
| MS (M/2 + H)⁺ Observ. | 1050 |
| Retention Time | 1.95 min |

LC Condition

| | |
|---|---|
| Solvent A | 90% Water-10% Methanol-0.1% TFA |
| Solvent B | 10% Water-90% Methanol-0.1% TFA |
| Start % B | 70 |
| Final % B | 100 |
| Gradient Time | 3 min |
| Flow Rate | 1 mL/min |
| Wavelength | 220 |
| Temperature | 40° C. |
| Column | PHENOMENEX-LUNA 2.0 × 30 mm 3 um |

Compound 1007

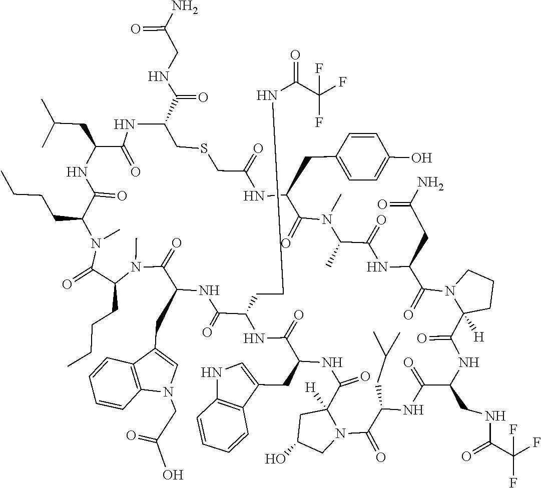 [SMILES: CCCC[C@H]1C(=O)N(C)[C@@H](CCCC)C(=O)N[C@@H](CC(C)C)C(=O)N[C@H](C(=O)NCC(N)=O)CSCC(=O)NC[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)C(F)(F)F)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(F)(F)F)C(=O)N[C@@H](Cc2cn(CC(=O)O)c3ccccc23)C(=O)N1C]

Agents Used

| | |
|---|---|
| Starting Material | Example 1 |
| Electrophile | 2,2,2-Trifluoro-1-(1H-imidazol-1-yl)ethanone |

MS

| | |
|---|---|
| MS (M/2 + H)⁺ Calcd. | 1040 |
| MS (M/2 + H)⁺ Observ. | 1040 |
| Retention Time | 2.35 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1008

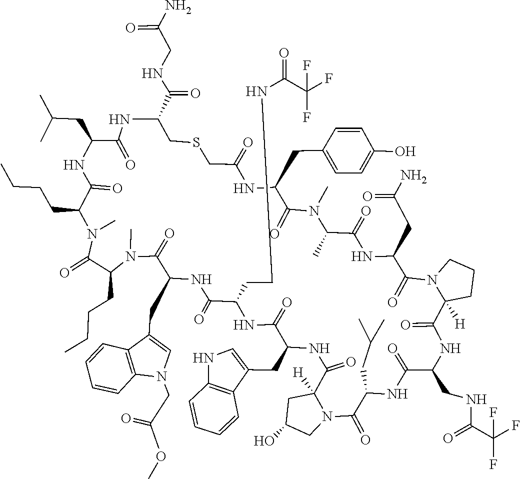 [SMILES: CCCC[C@H]1C(=O)N(C)[C@@H](CCCC)C(=O)N[C@@H](CC(C)C)C(=O)N[C@H](C(=O)NCC(N)=O)CSCC(=O)NC[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)C(F)(F)F)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(F)(F)F)C(=O)N[C@@H](Cc2cn(CC(=O)OC)c3ccccc23)C(=O)N1C]

Agents Used

| | |
|---|---|
| Starting Material | Example 1 |
| Electrophile | 2,2,2-Trifluoro-1-(1H-imidazol-1-yl)ethanone |

MS

| | |
|---|---|
| MS (M/2 + H)⁺ Calcd. | 1047 |
| MS (M/2 + H)⁺ Observ. | 1047 |
| Retention Time | 2.92 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1009

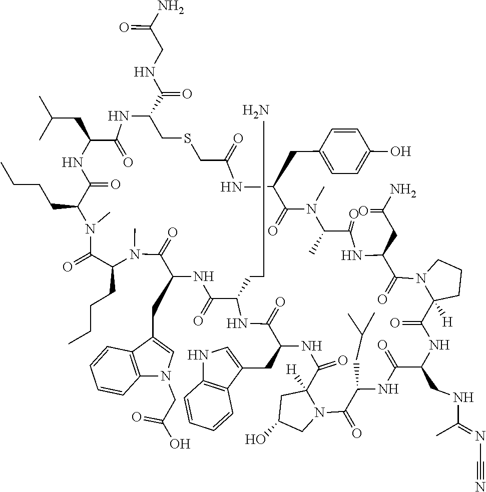 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN/C(C)=N/C#N)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | 2,2,2-Trifluoro-1-(1H-imidazol-1-yl)ethanone |

MS

| | |
|---|---|
| MS (M/2 + H)⁺ Calcd. | 977 |
| MS (M/2 + H)⁺ Observ. | 977 |
| Retention Time | 2.24 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1010

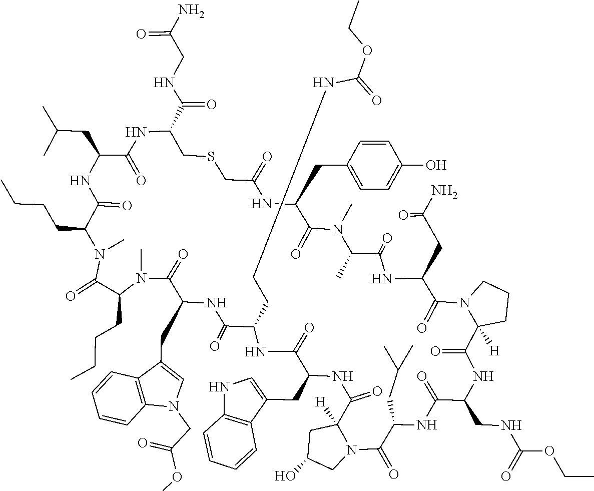

Agents Used

| | |
|---|---|
| Starting Material | Example 1 |
| Electrophile | Ethyl Chloroformate |

MS

| | |
|---|---|
| MS (M/2 + H)⁺ Calcd. | 1023 |
| MS (M/2 + H)⁺ Observ. | 1023 |
| Retention Time | 1.69 min |

LC Condition

| | |
|---|---|
| Solvent A | 5% ACN:95% Water:10 mM Ammonium Acetate |
| Solvent B | 95% ACN:5% water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2 , 3 u |

Compound 1012

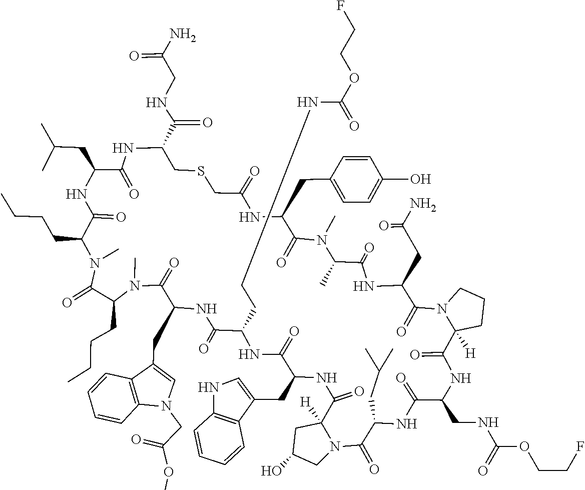

Agents Used

| | |
|---|---|
| Starting Material | Example 1 |
| Electrophile | 2-Fluoroethyl Chloroformate |

MS

| | |
|---|---|
| MS (M/2 + H)⁺ Calcd. | 1041 |
| MS (M/2 + H)⁺ Observ. | 1042 |
| Retention Time | 1.75 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1013

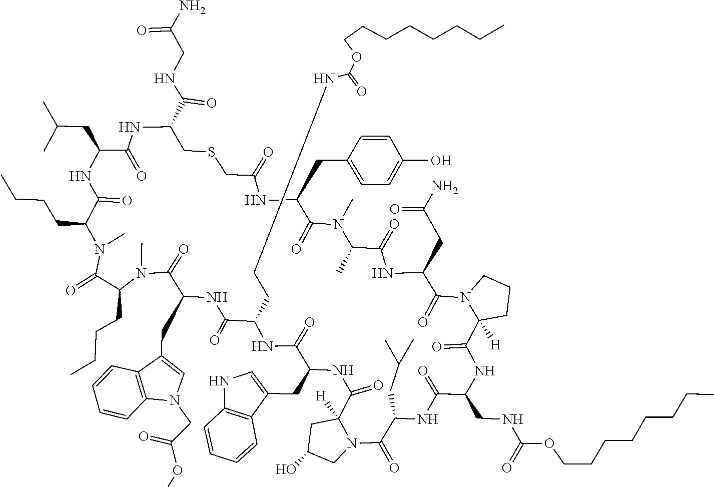 [SMILES: CCCCCCCCOC(=O)NCC[C@@H]1NC(=O)[C@H](Cc2c[nH]c3ccccc23)NC(=O)[C@@H]2C[C@@H](O)CN2C(=O)[C@H](CC(C)C)NC(=O)[C@H](CNC(=O)OCCCCCCCC)NC(=O)[C@@H]2CCCN2C(=O)[C@H](CC(N)=O)NC(=O)[C@H](C)N(C)C(=O)[C@H](Cc2ccc(O)cc2)NC(=O)CSC[C@@H](C(=O)NCC(N)=O)NC(=O)[C@H](CC(C)C)NC(=O)[C@H](CCCC)N(C)C(=O)[C@H](CCCC)N(C)C(=O)[C@H](Cc2cn(CC(=O)OC)c3ccccc23)NC1=O]

Agents Used

| | |
|---|---|
| Starting Material | Example 1 |
| Electrophile | Octyl Chloroformate |

MS

| | |
|---|---|
| MS (M/2 + H)⁺ Calcd. | 1107 |
| MS (M/2 + H)⁺ Observ. | 1108 |
| Retention Time | 2.32 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1014

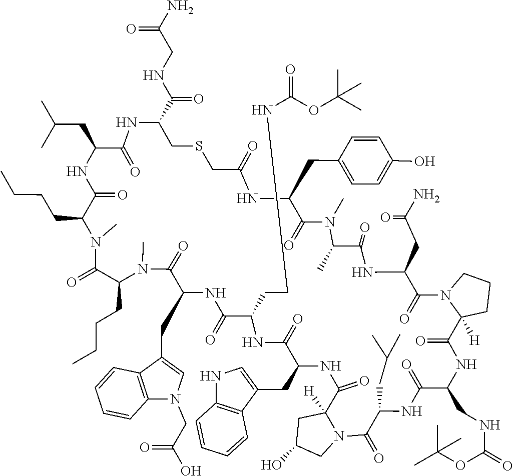

Agents Used

| | |
|---|---|
| Starting Material | Example 1 |
| Electrophile | Di-tert-butyl Dicarbonate |

MS

| | |
|---|---|
| MS (M/2 + H)⁺ Calcd. | 1044 |
| MS (M/2 + H)⁺ Observ. | 1044 |
| Retention Time | 1.56 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1015

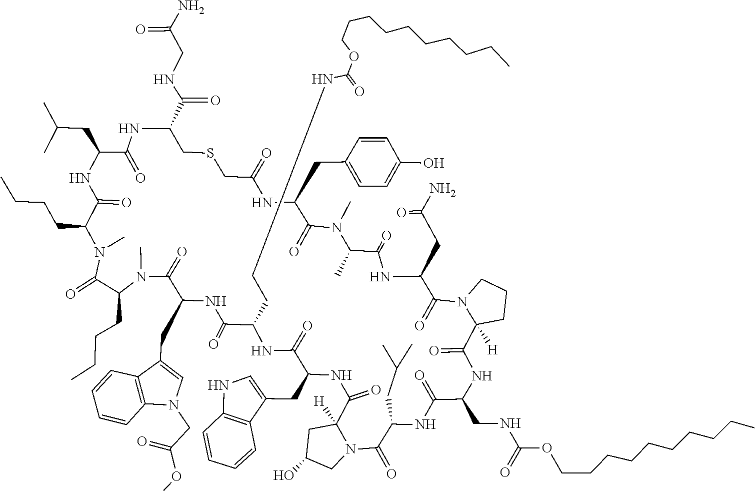 [SMILES: CCCCCCCCCCOC(=O)NCC[C@@H]1NC(=O)[C@H](Cc2c[nH]c3ccccc23)NC(=O)[C@@H]2C[C@@H](O)CN2C(=O)[C@H](CC(C)C)NC(=O)[C@H](CNC(=O)OCCCCCCCCCC)NC(=O)[C@@H]2CCCN2C(=O)[C@H](CC(N)=O)NC(=O)[C@H](C)N(C)C(=O)[C@H](Cc2ccc(O)cc2)NC(=O)CSC[C@@H](C(=O)NCC(N)=O)NC(=O)[C@H](CC(C)C)NC(=O)[C@H](CCCC)N(C)C(=O)[C@H](CCCC)N(C)C(=O)[C@H](Cc2cn(CC(=O)OC)c3ccccc23)NC1=O]

Agents Used

| | |
|---|---|
| Starting Material | Example 2 |
| Electrophile | Decyl Chloroformate |

MS

| | |
|---|---|
| MS (M/2 + H)⁺ Calcd. | 1135 |
| MS (M/2 + H)⁺ Observ. | 1135 |
| Retention Time | 2.07 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 50 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1016

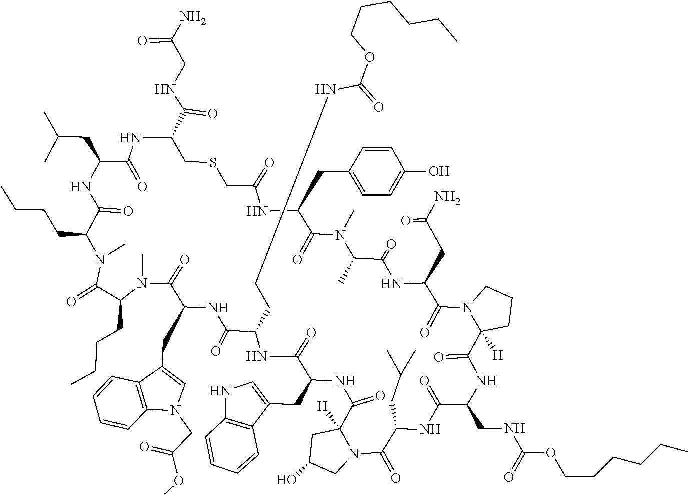 [SMILES: CCCCCCOC(=O)NCC[C@@H]1NC(=O)[C@H](Cc2c[nH]c3ccccc23)NC(=O)[C@@H]2C[C@@H](O)CN2C(=O)[C@H](CC(C)C)NC(=O)[C@H](CNC(=O)OCCCCCC)NC(=O)[C@@H]2CCCN2C(=O)[C@H](CC(N)=O)NC(=O)[C@H](C)N(C)C(=O)[C@H](Cc2ccc(O)cc2)NC(=O)CSC[C@@H](C(=O)NCC(N)=O)NC(=O)[C@H](CC(C)C)NC(=O)[C@H](CCCC)N(C)C(=O)[C@H](CCCC)N(C)C(=O)[C@H](Cc2cn(CC(=O)OC)c3ccccc23)NC1=O]

Agents Used

| | |
|---|---|
| Starting Material | Example 2 |
| Electrophile | Hexyl Chloroformate |

MS

| | |
|---|---|
| MS (M/2 + H)⁺ Calcd. | 1079 |
| MS (M/2 + H)⁺ Observ. | 1079 |
| Retention Time | 1.42 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 50 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1017

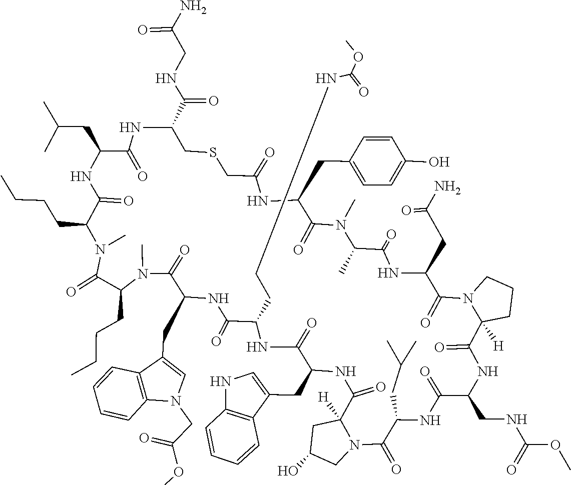 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)OC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)OC)C(=O)N[C@@H](Cc2cn(CC(=O)OC)c3ccccc23)C(=O)N1C]

Agents Used

| | |
|---|---|
| Starting Material | Example 2 |
| Electrophile | Methyl Chloroformate |

MS

| | |
|---|---|
| MS (M/2 + H)⁺ Calcd. | 1009 |
| MS (M/2 + H)⁺ Observ. | 1009 |
| Retention Time | 1.96 min |

LC Condition

| | |
|---|---|
| Solvent A | 90% Water-10% Methanol-0.1% TFA |
| Solvent B | 10% Water-90% Methanol-0.1% TFA |
| Start % B | 50 |
| Final % B | 100 |
| Gradient Time | 2 min |
| Flow Rate | 1 mL/min |
| Wavelength | 220 |
| Temperature | 40° C. |
| Column | PHENOMENEX-LUNA 2.0 × 30 mm 3 um |

Compound 1018

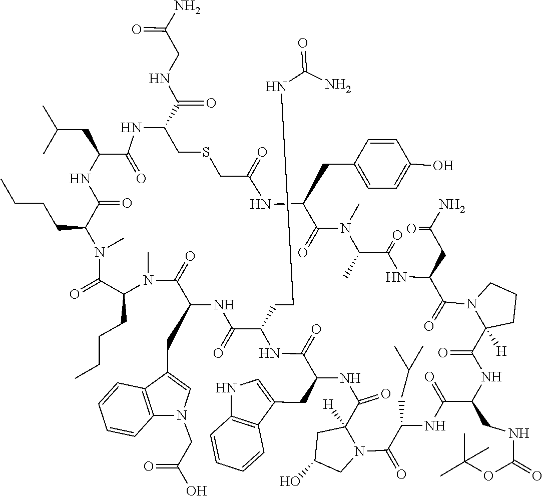 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)OC(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(N)=O)C(=O)N[C@@H](Cc2cn(CC(=O)O)c3ccccc23)C(=O)N1C]

Agents Used

| | |
|---|---|
| Starting Material | Example 1 |
| Electrophile | Di-tert-butyl Dicarbonate |

MS

| | |
|---|---|
| MS (M/2 + H)⁺ Calcd. | 1016 |
| MS (M/2 + H)⁺ Observ. | 1016 |
| Retention Time | 1.51 min |

LC Condition

| | |
|---|---|
| Solvent A | 5:95 ACN:water with 0.1% trifluoroacetic acid |
| Solvent B | 95:5 ACN:water with 0.1% trifluoroacetic acid |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1019

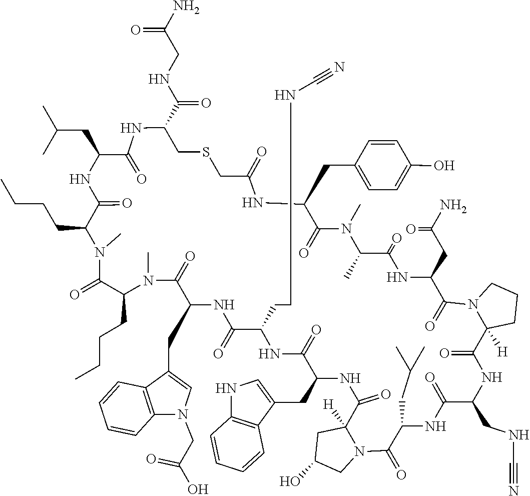 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC#N)C(=O)N[C@@H](CC(C)C)C(=O)N2C[C@H](O)C[C@H]2C(=O)N[C@@H](Cc2c[nH]c3ccccc23)C(=O)N[C@@H](CCNC#N)C(=O)N[C@@H](Cc2cn(CC(=O)O)c3ccccc23)C(=O)N1C]

Agents Used

| | |
|---|---|
| Starting Material | Example 1 |
| Electrophile | Cyanic Bromide |

MS

| | |
|---|---|
| MS (M/2 + H)⁺ Calcd. | 969 |
| MS (M/2 + H)⁺ Observ. | 969 |
| Retention Time | 1.41 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1020

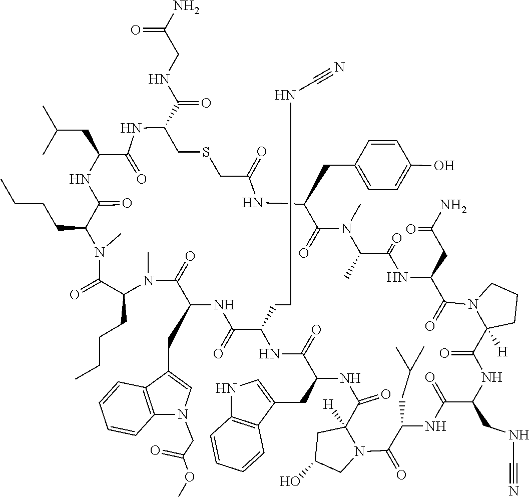 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC#N)C(=O)N[C@@H](CC(C)C)C(=O)N2C[C@H](O)C[C@H]2C(=O)N[C@@H](Cc2c[nH]c3ccccc23)C(=O)N[C@@H](CCNC#N)C(=O)N[C@@H](Cc2cn(CC(=O)OC)c3ccccc23)C(=O)N1C]

Agents Used

| | |
|---|---|
| Starting Material | Example 1 |
| Electrophile | Cyanic Bromide |

MS

| | |
|---|---|
| MS (M/2 + H)⁺ Calcd. | 976 |
| MS (M/2 + H)⁺ Observ. | 976 |
| Retention Time | 1.89 min |

LC Condition

| | |
|---|---|
| Solvent A | 5:95 ACN:water with 0.1% trifluoroacetic acid |
| Solvent B | 95:5 ACN:water with 0.1% trifluoroacetic acid |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1021

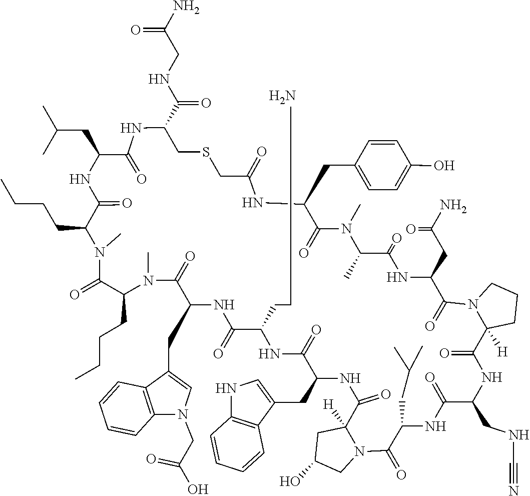 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC#N)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | (E)-methyl N-cyanoacetimidate |

MS

| | |
|---|---|
| MS (M/2 + H)⁺ Calcd. | 957 |
| MS (M/2 + H)⁺ Observ. | 957 |
| Retention Time | 1.47 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1022

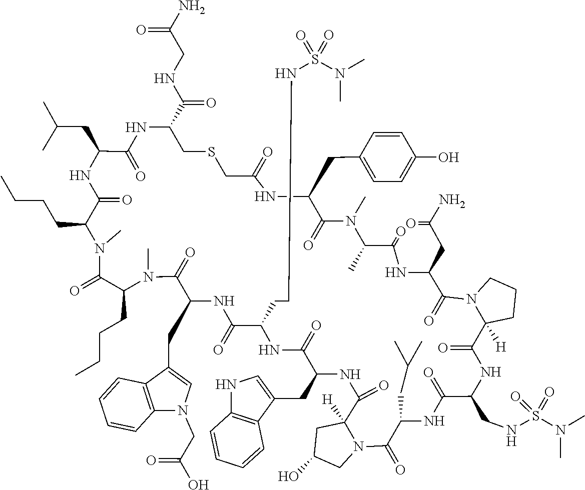 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNS(=O)(=O)N(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNS(=O)(=O)N(C)C)C(=O)N[C@@H](Cc2cn(CC(=O)O)c3ccccc23)C(=O)N1C]

Agents Used

| | |
|---|---|
| Starting Material | Example 1 |
| Electrophile | Dimethylsulfamoyl Chloride |

MS

| | |
|---|---|
| MS (M/2 + H)⁺ Calcd. | 1051 |
| MS (M/2 + H)⁺ Observ. | 1051 |
| Retention Time | 1.59 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1023

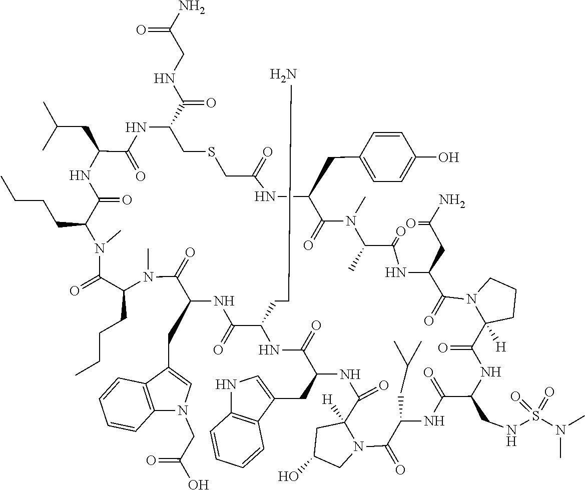 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNS(=O)(=O)N(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | Dimethylsulfamoyl Chloride |

MS

| | |
|---|---|
| MS (M/2 + H)⁺ Calcd. | 997 |
| MS (M/2 + H)⁺ Observ. | 997 |
| Retention Time | 1.48 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1024

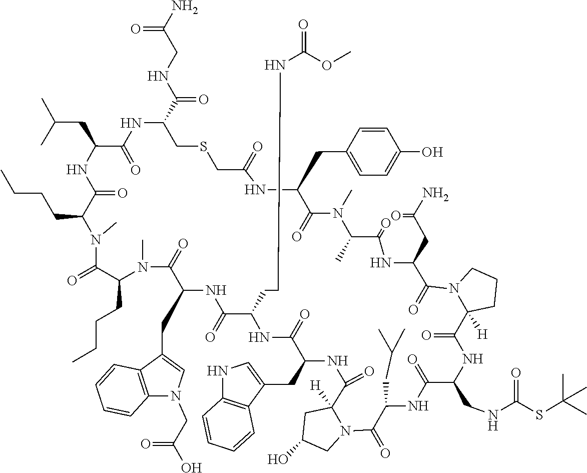 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)SC(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)OC)C(=O)N[C@@H](Cc2cn(CC(=O)O)c3ccccc23)C(=O)N1C]

Agents Used

| | |
|---|---|
| Starting Material | Example 1 |
| Electrophile | S-tert-butyl Carbonochloridothioate |

MS

| | |
|---|---|
| MS (M/2 + H)⁺ Calcd. | 1031 |
| MS (M/2 + H)⁺ Observ. | 1031 |
| Retention Time | 2.08 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1025

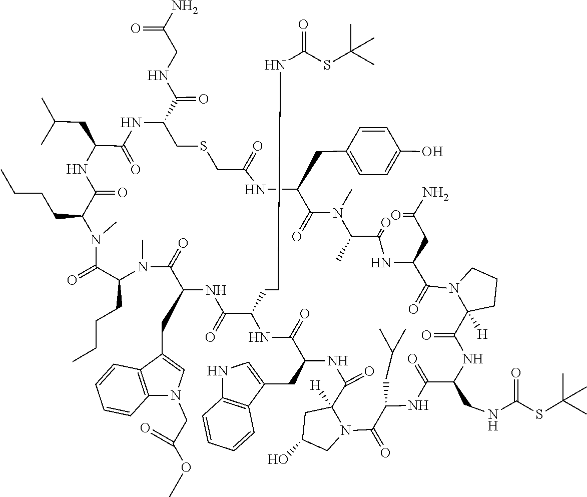 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)SC(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)SC(C)(C)C)C(=O)N[C@@H](Cc2cn(CC(=O)OC)c3ccccc23)C(=O)N1C]

Agents Used

| | |
|---|---|
| Starting Material | Example 1 |
| Electrophile | S-tert-butyl Carbonochloridothioate |

MS

| | |
|---|---|
| MS (M/2 + H)⁺ Calcd. | 1067 |
| MS (M/2 + H)⁺ Observ. | 1067 |
| Retention Time | 2.14 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1026

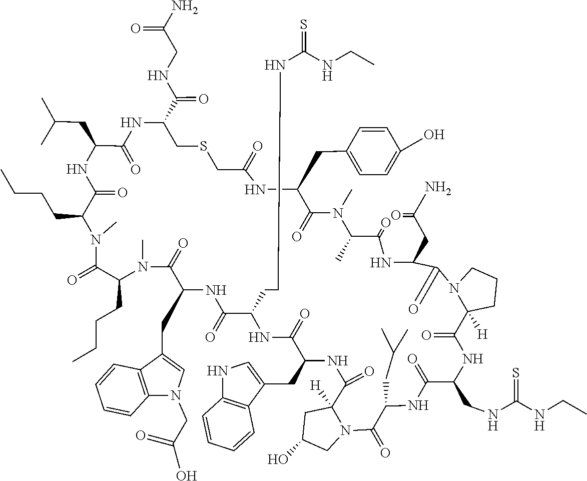 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=S)NCC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=S)NCC)C(=O)N[C@@H](Cc2cn(CC(=O)O)c3ccccc23)C(=O)N1C]

Agents Used

| | |
|---|---|
| Starting Material | Example 1 |
| Electrophile | Isothiocyanatoethane |

MS

| | |
|---|---|
| MS (M/2 + H)⁺ Calcd. | 1031 |
| MS (M/2 + H)⁺ Observ. | 1031 |
| Retention Time | 2.20 min |

LC Condition

| | |
|---|---|
| Solvent A | 5:95 acetonitrile:water with 10 mM ammonium acetate |
| Solvent B | 95:5 acetonitrile:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1027

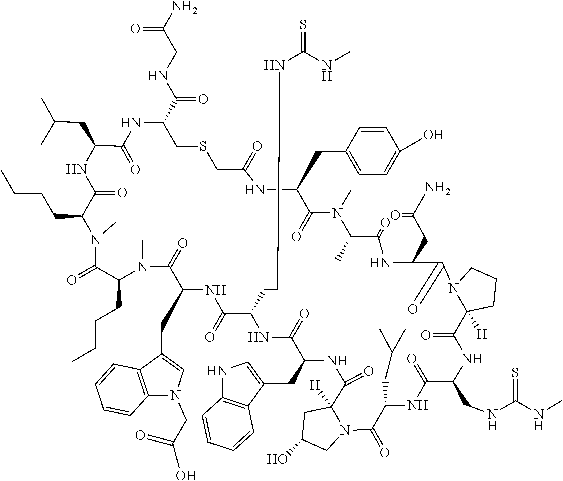 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@H](CC(N)=O)C(=O)N2CCC[C@H]2C(=O)N[C@@H](CNC(=S)NC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=S)NC)C(=O)N[C@@H](Cc2cn(CC(=O)O)c3ccccc23)C(=O)N1C]

Agents Used

| | |
|---|---|
| Starting Material | Example 1 |
| Electrophile | Isothiocyanatomethane |

MS

| | |
|---|---|
| MS (M/2 + H)⁺ Calcd. | 1017 |
| MS (M/2 + H)⁺ Observ. | 1017 |
| Retention Time | 1.78 min |

LC Condition

| | |
|---|---|
| Solvent A | 5:95 ACN:water with 10 mM ammonium acetate |
| Solvent B | 95:5 acetonitrile:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1028

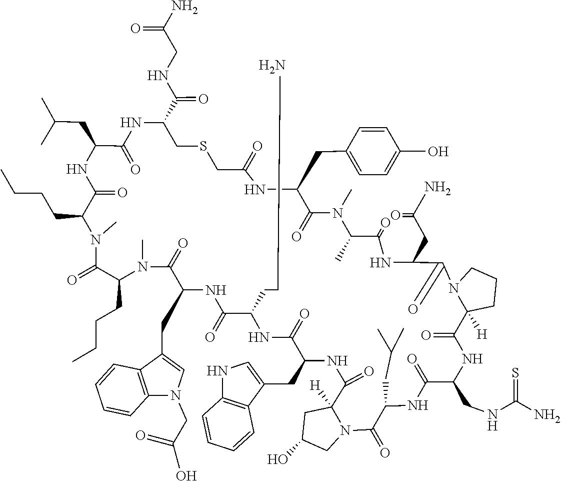 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@H](CC(N)=O)C(=O)N2CCC[C@H]2C(=O)N[C@@H](CNC(N)=S)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | (Isothiocyanatomethanetriyl)tribenzene |

MS

| | |
|---|---|
| MS (M/2 + H)⁺ Calcd. | 974 |
| MS (M/2 + H)⁺ Observ. | 974 |
| Retention Time | 1.66 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1029

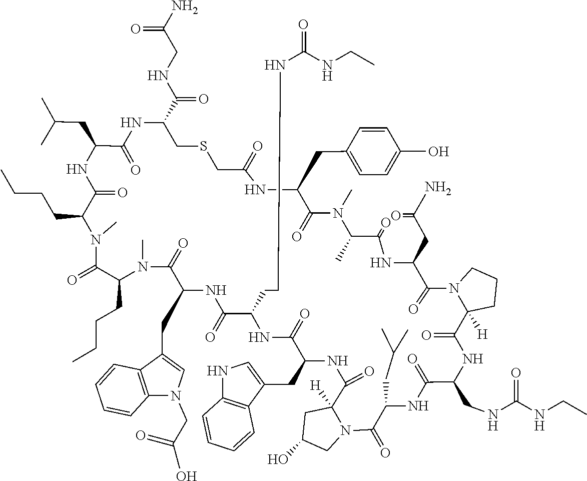 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)NCC)C(=O)N[C@@H](Cc2cn(CC(=O)O)c3ccccc23)C(=O)N1C]

Agents Used

| | |
|---|---|
| Starting Material | Example 1 |
| Electrophile | Isocyanatoethane |

MS

| | |
|---|---|
| MS (M/2 + H)⁺ Calcd. | 1015 |
| MS (M/2 + H)⁺ Observ. | 1015 |
| Retention Time | 1.41 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1030

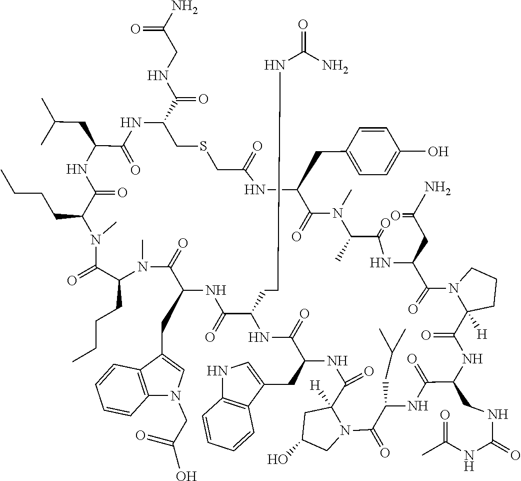 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NC(C)=O)C(=O)N[C@@H](CC(C)C)C(=O)N2C[C@H](O)C[C@H]2C(=O)N[C@@H](Cc2c[nH]c3ccccc23)C(=O)N[C@@H](CCNC(N)=O)C(=O)N[C@@H](Cc2cn(CC(=O)O)c3ccccc23)C(=O)N1C]

Agents Used

| | |
|---|---|
| Starting Material | Example 1 |
| Electrophile | Acetyl Isocyanate |

MS

| | |
|---|---|
| MS (M/2 + H)⁺ Calcd. | 1008 |
| MS (M/2 + H)⁺ Observ. | 1008 |
| Retention Time | 1.44 min |

LC Condition

| | |
|---|---|
| Solvent A | 5% ACN:95% Water:10 mM Ammonium Acetate |
| Solvent B | 95% ACN:5% Water:10 mM Ammonium Acetate |
| Start % B | 0 |
| Final % B | 100 |
| Gradient Time | 2 min |
| Flow Rate | 1 mL/min |
| Wavelength | 220 |
| Temperature | 40° C. |
| Column | Phenomenex LUNA C18, 30 × 2, 3 u |

Compound 1031

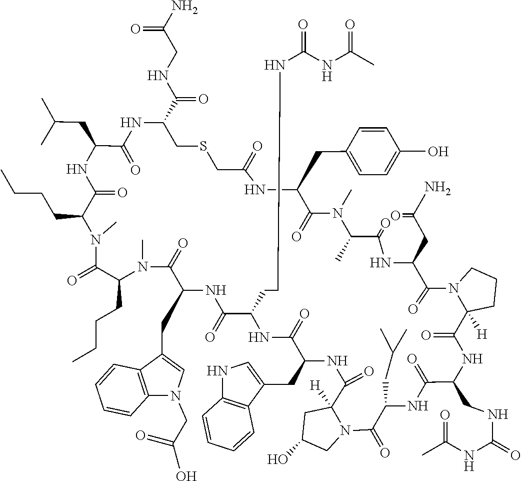 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NC(C)=O)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)NC(C)=O)C(=O)N[C@@H](Cc2cn(CC(=O)O)c3ccccc23)C(=O)N1C]

Agents Used

| | |
|---|---|
| Starting Material | Example 1 |
| Electrophile | Acetyl Isocyanate |

MS

| | |
|---|---|
| MS (M/2 + H)⁺ Calcd. | 1029 |
| MS (M/2 + H)⁺ Observ. | 1029 |
| Retention Time | 1.84 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1032

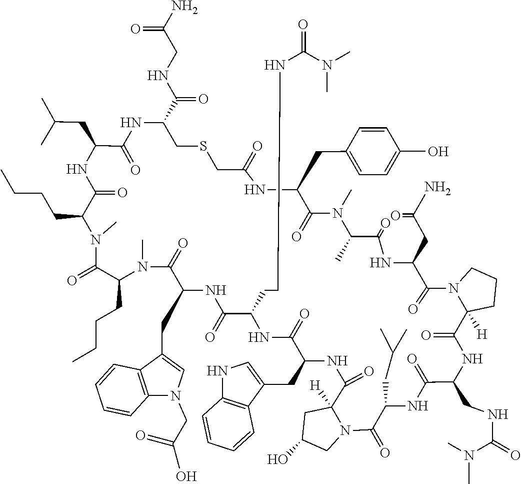 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)N(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)N(C)C)C(=O)N[C@@H](Cc2cn(CC(=O)O)c3ccccc23)C(=O)N1C]

Agents Used

| | |
|---|---|
| Starting Material | Example 1 |
| Electrophile | Dimethylcarbamic Chloride |

MS

| | |
|---|---|
| MS (M/2 + H)⁺ Calcd. | 1015 |
| MS (M/2 + H)⁺ Observ. | 1015 |
| Retention Time | 1.72 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1033

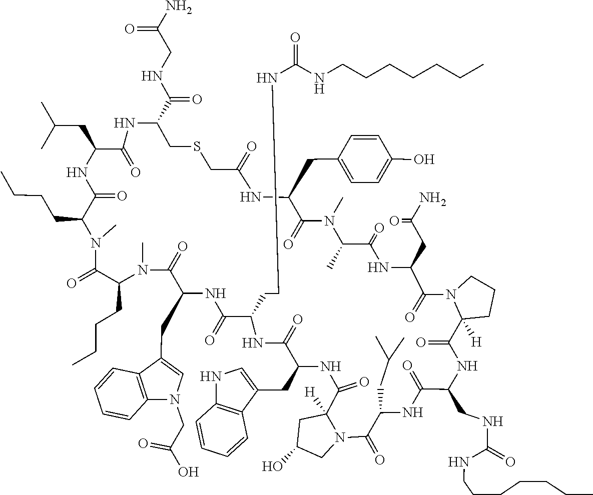 [SMILES: CCCCCCCNC(=O)NCC[C@@H]1NC(=O)[C@H](Cc2c[nH]c3ccccc23)NC(=O)[C@@H]2C[C@@H](O)CN2C(=O)[C@H](CC(C)C)NC(=O)[C@H](CNC(=O)NCCCCCCC)NC(=O)[C@@H]2CCCN2C(=O)[C@H](CC(N)=O)NC(=O)[C@H](C)N(C)C(=O)[C@H](Cc2ccc(O)cc2)NC(=O)CSC[C@@H](C(=O)NCC(N)=O)NC(=O)[C@H](CC(C)C)NC(=O)[C@H](CCCC)N(C)C(=O)[C@H](CCCC)N(C)C(=O)[C@H](Cc2cn(CC(=O)O)c3ccccc23)NC1=O]

Agents Used

| | |
|---|---|
| Starting Material | Example 1 |
| Electrophile | 1-Isocyanatoheptane |

MS

| | |
|---|---|
| MS (M/2 + H)⁺ Calcd. | 1085 |
| MS (M/2 + H)⁺ Observ. | 1085 |
| Retention Time | 1.96 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1034

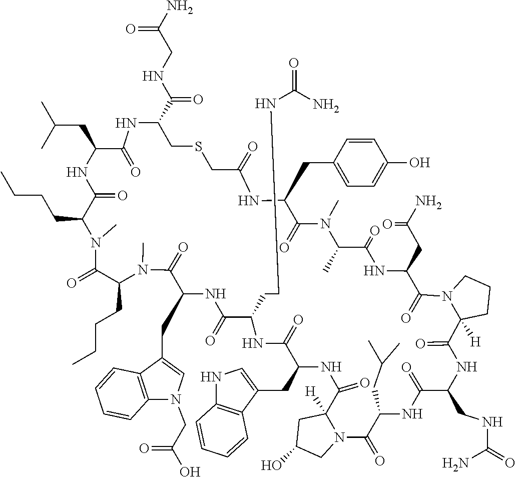 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(N)=O)C(=O)N[C@@H](CC(C)C)C(=O)N2C[C@H](O)C[C@H]2C(=O)N[C@@H](Cc2c[nH]c3ccccc23)C(=O)N[C@@H](CCNC(N)=O)C(=O)N[C@@H](Cc2cn(CC(=O)O)c3ccccc23)C(=O)N1C]

Agents Used

| | |
|---|---|
| Starting Material | Example 1 |
| Electrophile | Acetyl Isocyanate |

MS

| | |
|---|---|
| MS (M/2 + H)⁺ Calcd. | 987 |
| MS (M/2 + H)⁺ Observ. | 986 |
| Retention Time | 1.52 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1035

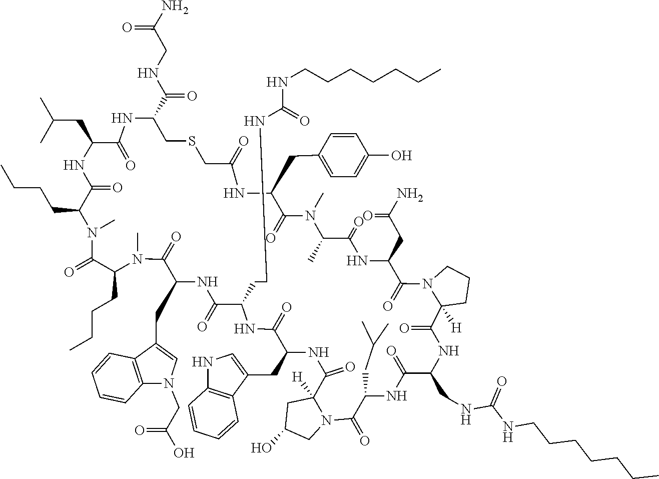

Agents Used

| | |
|---|---|
| Starting Material | Example 1 |
| Electrophile | 1-Isocyanatoheptane |

MS

| | |
|---|---|
| MS (M/2 + H)⁺ Calcd. | 1085 |
| MS (M/2 + H)⁺ Observ. | 1085 |
| Retention Time | 2.03 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1036

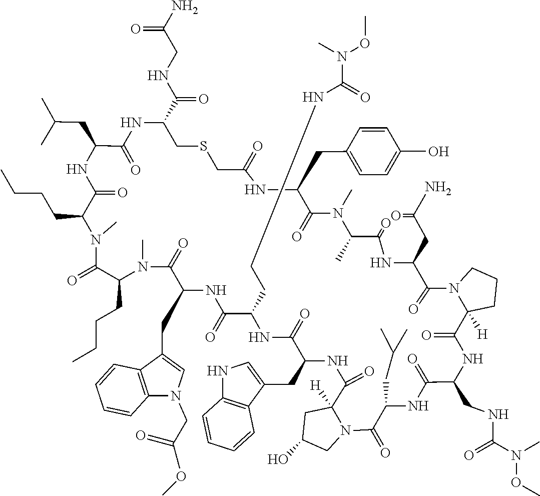 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)N(C)OC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)N(C)OC)C(=O)N[C@@H](Cc2cn(CC(=O)OC)c3ccccc23)C(=O)N1C]

Agents Used

| | |
|---|---|
| Starting Material | Example 2 |
| Electrophile | N-methoxy-N-Methylcarbamoyl Chloride |

MS

| | |
|---|---|
| MS (M/2 + H)⁺ Calcd. | 1038 |
| MS (M/2 + H)⁺ Observ. | 1038 |
| Retention Time | 1.91 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm particles |

Compound 1037

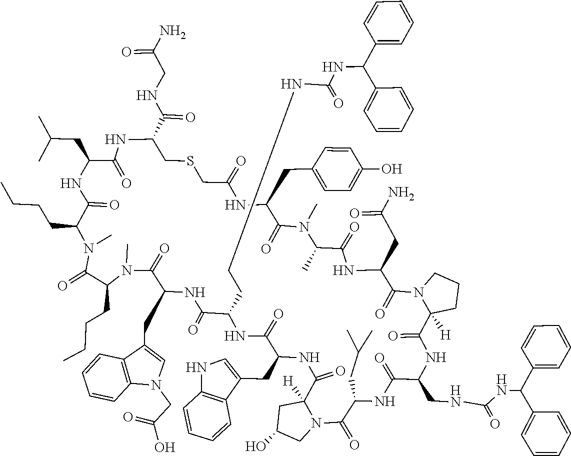 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NC(c2ccccc2)c2ccccc2)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)NC(c2ccccc2)c2ccccc2)C(=O)N[C@@H](Cc2cn(CC(=O)O)c3ccccc23)C(=O)N1C]

Agents Used

| | |
|---|---|
| Starting Material | Example 1 |
| Electrophile | (Isocyanatomethylene)dibenzene |

MS

| | |
|---|---|
| MS (M/2 + H)⁺ Calcd. | 1053 |
| MS (M/2 + H)⁺ Observ. | 1053 |
| Retention Time | 2.05 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1038

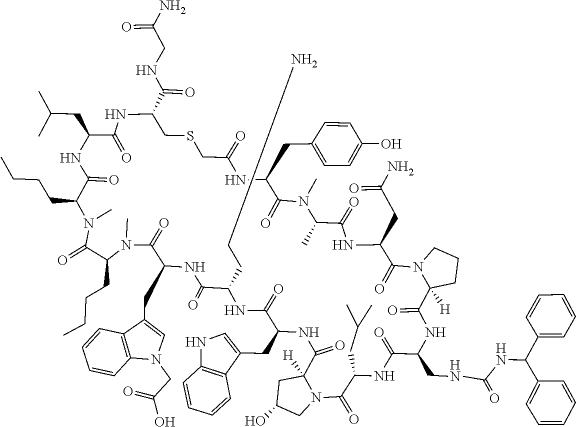 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NC(c2ccccc2)c2ccccc2)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | (Isocyanatomethylene)dibenzene |

MS

| | |
|---|---|
| MS (M/2 + H)⁺ Calcd. | 1049 |
| MS (M/2 + H)⁺ Observ. | 1049 |
| Retention Time | 1.98 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1039

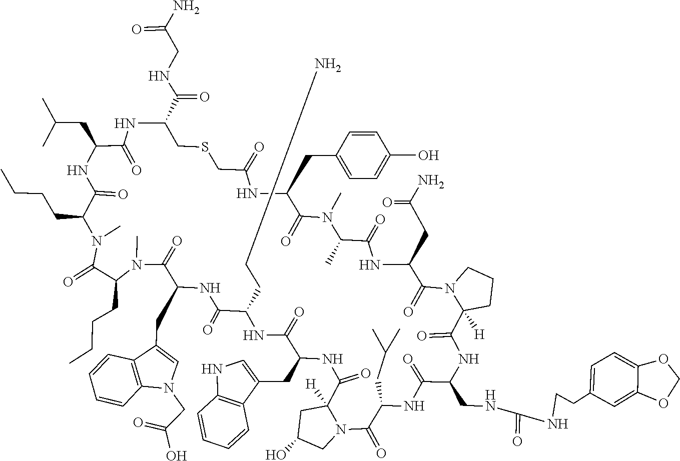 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCCc2ccc3c(c2)OCO3)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | 5-(2-Isocyanatoethyl)benzo[d][1,3]dioxole |

MS

| | |
|---|---|
| MS (M/2 + H)⁺ Calcd. | 1040 |
| MS (M/2 + H)⁺ Observ. | 1040 |
| Retention Time | 1.68 min |

LC Condition

| | |
|---|---|
| Solvent A | 5:95 ACN:water with 10 mM ammonium acetate |
| Solvent B | 95:5 acetonitrile:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1040

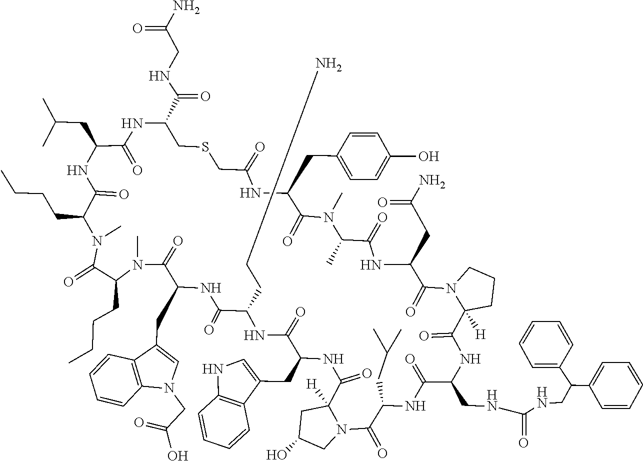 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCC(c2ccccc2)c2ccccc2)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | (2-Isocyanatoethane-1,1-diyl)dibenzene |

MS

| | |
|---|---|
| MS (M/2 + H)⁺ Calcd. | 1056 |
| MS (M/2 + H)⁺ Observ. | 1056 |
| Retention Time | 1.83 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1041

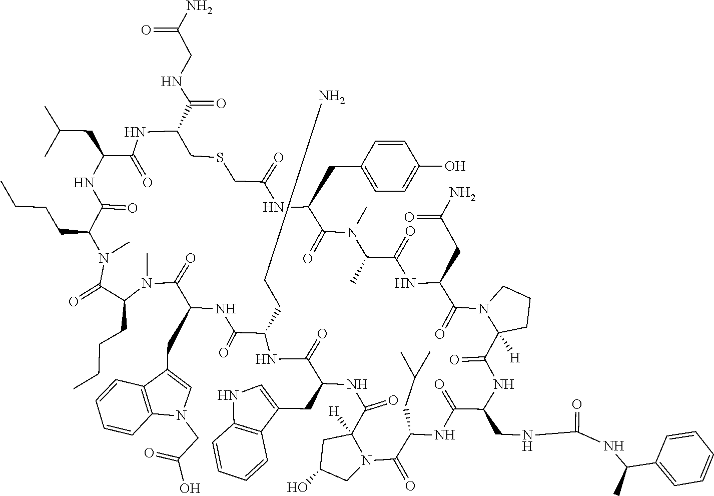 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)N[C@H](C)c2ccccc2)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | (R)-(1-Isocyanatoethyl)benzene |

MS

| | |
|---|---|
| MS (M/2 + H)⁺ Calcd. | 1018 |
| MS (M/2 + H)⁺ Observ. | 1018 |
| Retention Time | 1.71 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μm particles |

Compound 1042

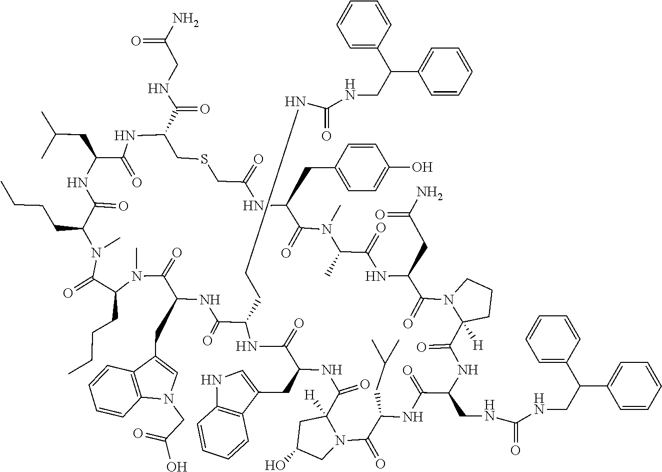 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCC(c2ccccc2)c2ccccc2)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)NCC(c2ccccc2)c2ccccc2)C(=O)N[C@@H](Cc2cn(CC(=O)O)c3ccccc23)C(=O)N1C]

Agents Used

| | |
|---|---|
| Starting Material | Example 1 |
| Electrophile | (2-Isocyanatoethane-1,1-diyl)dibenzene |

MS

| | |
|---|---|
| MS (M/2 + H)⁺ Calcd. | 1167 |
| MS (M/2 + H)⁺ Observ. | 1167 |
| Retention Time | 2.10 min |

LC Condition

| | |
|---|---|
| Solvent A | 5:95 acetonitrile:water with 10 mM ammonium acetate |
| Solvent B | 95:5 acetonitrile:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1043

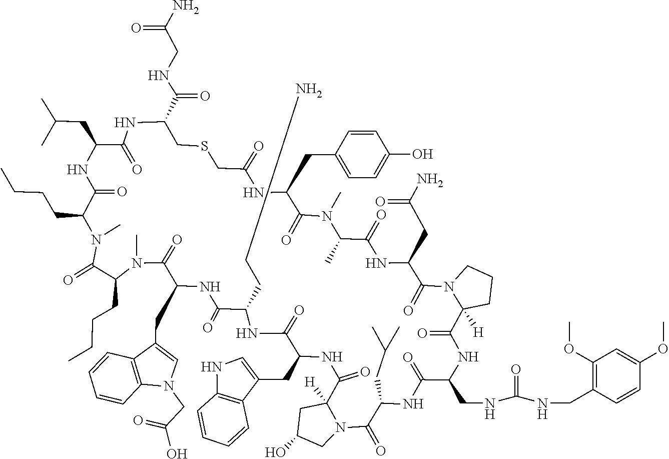 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCc2ccc(OC)cc2OC)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | 1-(Isocyanatomethyl)-2,4-dimethoxybenzene |

MS

| | |
|---|---|
| MS (M/2 + H)⁺ Calcd. | 1041 |
| MS (M/2 + H)⁺ Observ. | 1041 |
| Retention Time | 1.80 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1044

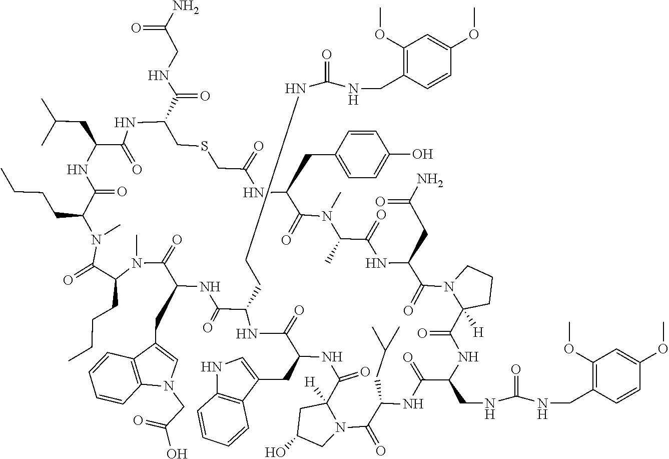 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)NCc2ccc(OC)cc2OC)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)NCc2ccc(OC)cc2OC)C(=O)N[C@@H](Cc2cn(CC(=O)O)c3ccccc23)C(=O)N1C]

Agents Used

| | |
|---|---|
| Starting Material | Example 1 |
| Electrophile | 1-(Isocyanatomethyl)-2,4-dimethoxybenzene |

MS

| | |
|---|---|
| MS (M/2 + H)⁺ Calcd. | 1137 |
| MS (M/2 + H)⁺ Observ. | 1137 |
| Retention Time | 1.68 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1045

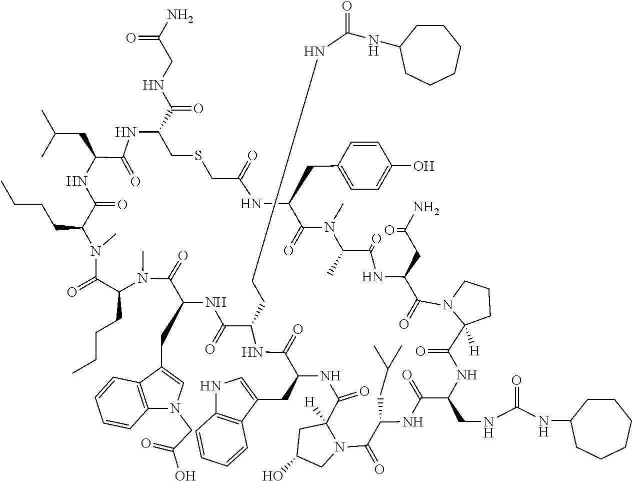

Agents Used

| | |
|---|---|
| Starting Material | Example 1 |
| Electrophile | Isocyanatocycloheptane |

MS

| | |
|---|---|
| MS (M/2 + H)⁺ Calcd. | 1083 |
| MS (M/2 + H)⁺ Observ. | 1083 |
| Retention Time | 1.78 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1046

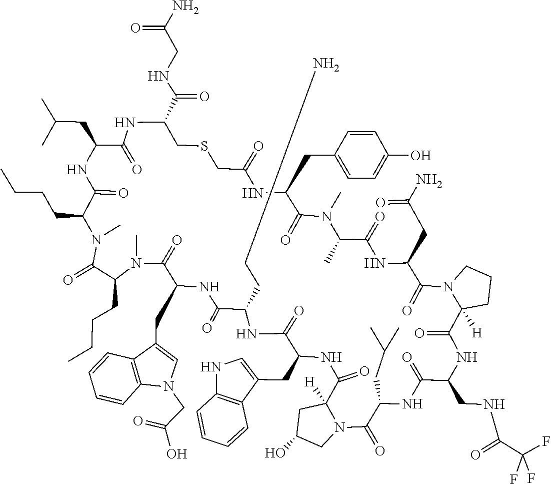 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)C(F)(F)F)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

Agents Used

| | |
|---|---|
| Starting Material | Example 1 |
| Electrophile | 2,2,2-Trifluoro-1-(4-isocyanatopiperidin-1-yl)ethan-1-one |

MS

| | |
|---|---|
| MS (M/2 + H)⁺ Calcd. | 1983 |
| MS (M/2 + H)⁺ Observ. | 1983 |
| Retention Time | 1.65 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1047

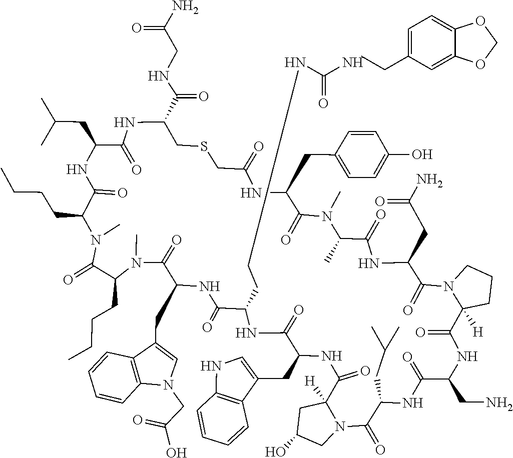

Agents Used

| | |
|---|---|
| Starting Material | Example 1 |
| Electrophile | 5-(2-Isocyanatoethyl)benzo[d][1,3]dioxole |

MS

| | |
|---|---|
| MS (M/2 + H)⁺ Calcd. | 1040 |
| MS (M/2 + H)⁺ Observ. | 1040 |
| Retention Time | 1.65 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

Compound 1048

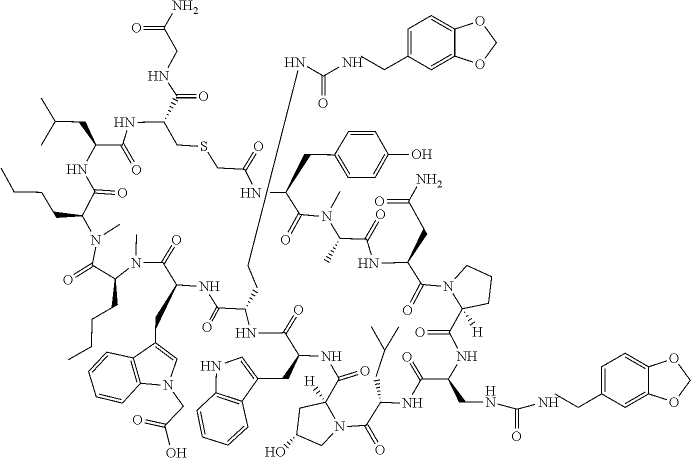

Agents Used

| | |
|---|---|
| Starting Material | Example 1 |
| Electrophile | 5-(2-Isocyanatoethyl)benzo[d][1,3]dioxole |

MS

| | |
|---|---|
| MS (M/2 + H)⁺ Calcd. | 1135 |
| MS (M/2 + H)⁺ Observ. | 1135 |
| Retention Time | 1.66 min |

LC Condition

| | |
|---|---|
| Solvent A | 5:95 ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 0.75 mL/min |
| Wavelength | 220 |
| Temperature | 70° C. |
| Column | Waters XBridge C18, 2.1 mm × 50 mm, 1.7 μm particles |

General Procedure for the Preparation of Methyl Ester of Compounds from the Corresponding Acid:

(Diazomethyl)trimethylsilane (2M in ether, 1-20 eq.) was added into a solution of acid (1 eq.) in THF or dioxane or DME with or without MeOH or EtOH. The reaction was stirred at room temperature for 0.5 to 48 hours, before the reaction was quenched with methanol or water. After all the solvents were removed under vacuum, the residue was purified by the preparative HPLC to provide the desired products.

Compound 2001

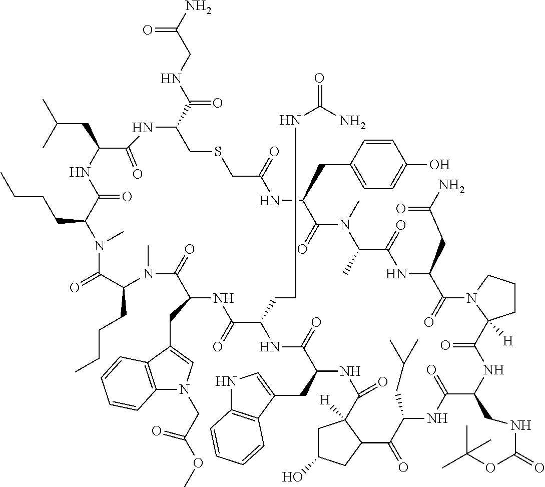 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)OC(C)(C)C)C(=O)N[C@@H](CC(C)C)C(=O)C2C[C@H](O)C[C@H]2C(=O)N[C@@H](Cc2c[nH]c3ccccc23)C(=O)N[C@@H](CCNC(N)=O)C(=O)N[C@@H](Cc2cn(CC(=O)OC)c3ccccc23)C(=O)N1C]

MS

| | |
|---|---|
| MS (M/2 + H)⁺ Calcd. | 1023 |
| MS (M/2 + H)⁺ Observ. | 1022 |
| Retention Time | 1.91 min |

LC Condition

| | |
|---|---|
| Solvent A | 5:95 |
| | ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 μ |
| | particles |

Compound 2002

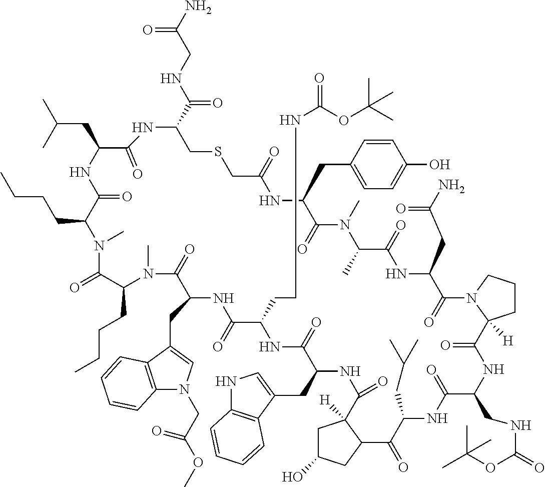 [SMILES: CCCC[C@H]1C(=O)N(C)[C@@H](CCCC)C(=O)N[C@@H](CC(C)C)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CNC(=O)OC(C)(C)C)C(=O)N[C@@H](CC(C)C)C(=O)C2C[C@H](O)C[C@H]2C(=O)N[C@@H](Cc2c[nH]c3ccccc23)C(=O)N[C@@H](CCNC(=O)OC(C)(C)C)C(=O)N[C@@H](Cc2cn(CC(=O)OC)c3ccccc23)C(=O)N1C]

MS

| | |
|---|---|
| MS (M/2 + H)⁺ Calcd. | 1051 |
| MS (M/2 + H)⁺ Observ. | 1051 |
| Retention Time | 2.09 min |

LC Condition

| | |
|---|---|
| Solvent A | 5:95 |
| | ACN:water with 10 mM ammonium acetate |
| Solvent B | 95:5 ACN:water with 10 mM ammonium acetate |
| Start % B | 0 |
| Final % B | 100 |
| Gradient Time | 3 min |
| Flow Rate | 1 mL/min |
| Wavelength | 220 |
| Temperature | 70° C. |
| Column | Waters XBridge C18, 2.1 mm × 50 mm, 1.7 um |
| | particles |

Compound 2003

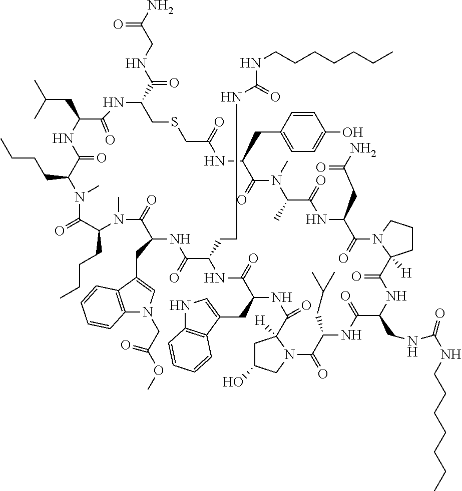 [SMILES: CCCCCCCNC(=O)NCC[C@@H]1NC(=O)[C@H](Cc2c[nH]c3ccccc23)NC(=O)[C@@H]2C[C@@H](O)CN2C(=O)[C@H](CC(C)C)NC(=O)[C@H](CNC(=O)NCCCCCCC)NC(=O)[C@@H]2CCCN2C(=O)[C@H](CC(N)=O)NC(=O)[C@H](C)N(C)C(=O)[C@H](Cc2ccc(O)cc2)NC(=O)CSC[C@@H](C(=O)NCC(N)=O)NC(=O)[C@H](CC(C)C)NC(=O)[C@H](CCCC)N(C)C(=O)[C@H](CCCC)N(C)C(=O)[C@H](Cc2cn(CC(=O)OC)c3ccccc23)NC1=O]

MS

| | |
|---|---|
| MS (M/2 + H)⁺ Calcd. | 1092 |
| MS (M/2 + H)⁺ Observ. | 1092 |
| Retention Time | 2.39 min |

LC Condition

| | |
|---|---|
| Solvent A | 5:95 |
| | acetonitrile:water with 10 mM ammonium acetate |
| Solvent B | 95:5 acetonitrile:water with 10 mM ammonium acetate |
| Start % B | 0 |
| Final % B | 100 |
| Gradient Time | 3 min |
| Flow Rate | 1 mL/min |
| Wavelength | 220 |
| Temperature | 50° C. |
| Column | Waters XBridge C18, 2.1 mm × 50 mm, 1.7 μm |
| | particles |

General Procedure for the Preparation of Alkyl Ester from the Corresponding Acid:

K₂CO₃ (1-50 eq.) was added into the solution of electrophile (1 eq.) and alkyl halide (1-10 eq.) in THF or dioxane or DME or DMF. The reaction was stirred at 85° C. for 0.5-48 hours. Then, NaH (1-50 eq.) was added and the resulting solution was heated at 85° C. for another 0.5-48 hours. After the solvents were removed under vacuum, the residue was purified by the preparative HPLC to give the desired methyl esters.

### General Procedures for Compounds:

All manipulations were performed under automation on a Prelude peptide synthesizer (Protein Technologies). All procedures unless noted were performed in a 40 ml reaction vessel fitted with a bottom frit. The vessel connects to a the Prelude peptide synthesizer through both the bottom and the top of the vessel. DMF and DCM can be added through the top of the vessel, which washes down the sides of the tube equally. The remaining reagents are added through the bottom of the tube and pass up through the frit to contact the resin. All solutions are removed through the bottom of the tube. “Periodic agitation” describes a brief pulse of N₂ gas through the bottom frit; the pulse lasts approximately 5 seconds and occurs every 30 seconds. 0.4 M of Chloroacetyl anhydride solutions in DMF were used within 5 days of preparation. Amino acid solutions were generally not used beyond three weeks from preparation. HATU solutions were used within 5 days of preparation. DMF=dimethylformamide; HATU=1-[Bis(dimethylamino)methylene]-1H-1,2,3-triazolo[4,5-b]pyridinium 3-oxide hexafluorophosphate; DIPEA=diisopropylethylamine; Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.53 mmol/g loading. Common amino acids used are listed with side-chain protecting groups indicated inside parenthesis: Fmoc-Ala-OH; Fmoc-Arg(Pbf)-OH; Fmoc-Asn(Trt)-OH; Fmoc-Asp(OtBu)-OH; Fmoc-Bzt-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH

The procedures of “Prelude Method A” describe an experiment performed on a 0.2 mmol scale, where the scale is determined by the amount of Rink linker bound to the resin. This scale corresponds to approximately 378 mg of the Rink-Merrifield resin described above. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus used the “Double-coupling procedure” described below. Coupling of chloroacetylchloride to the N-terminus of the peptide is described by the “Chloroacetyl chloride coupling procedure” detailed below.

### Resin-Swelling Procedure:

To a 40 mL polypropylene solid-phase reaction vessel was added Merrifield:Rink resin (378 mg, 0.200 mmol). The resin was washed (swelled) three times as follows: to the reaction vessel was added DMF (10.0 mL), upon which the mixture was periodically agitated for 10 minutes before the solvent was drained through the frit. The swelling was repeated one more time and the DMF was remove from the bottom of the vessel.

### Single-Coupling Procedure:

To the reaction vessel containing resin from the previous step was added piperidine:DMF (20:80 v/v, 6.0 mL). The mixture was periodically agitated for 5 minutes and then the solution was drained through the frit. To the reaction vessel was added piperidine:DMF (20:80 v/v, 6.0 mL). The mixture was periodically agitated for 5 minutes and then the solution was drained through the frit. The resin was washed successively six times as follows: for each wash, DMF (7.0 mL) was added through the top of the vessel and the resulting mixture was periodically agitated for 1 min. before the solution was drained through the frit. To the reaction vessel was added the amino acid (0.2M in DMF, 5.0 mL, 5 eq), then HATU (0.4M in DMF, 2.5 mL, 5 eq), and finally NMM (N-methylmorpholine, 0.8M in DMF, 0.25 mL, 10 eq). The mixture was periodically agitated for 2 hours, then the reaction solution was drained through the frit. The resin was washed successively five times as follows: for each wash, DMF (7.0 mL) was added through the top of the vessel and the resulting mixture was periodically agitated for 1 min. before the solution was drained through the frit. The resulting resin was used directly in the next step.

### Double-Coupling Procedure Used for Secondary Amino Acids:

After the first single-coupling procedure was finished it was repeated once more to make sure the coupling is completed.

### Custom Amino Acids-Coupling Procedure:

The procedure is same as Single-coupling procedure and Double-coupling procedure used for secondary amino acids described above.

### Chloroacetyl Chloride Coupling Procedure:

To the reaction vessel containing the resin from the previous step was added piperidine:DMF (20:80 v/v, 6.0 mL). The mixture was periodically agitated for 5 minutes and then the solution was drained through the frit. To the reaction vessel was added piperidine:DMF (20:80 v/v, 6.0 mL). The mixture was periodically agitated for 5 minutes and then the solution was drained through the frit. The resin was washed successively six times as follows: for each wash, DMF (7.0 mL) was added through the top of the vessel and the resulting mixture was periodically agitated for 1 min. before the solution was drained through the frit. To the reaction vessel was added chloroacetyl chloride (0.4M in DMF, 8 mL, 16 eq) and then NMM (N-methylmorpholine, 0.8M in DMF, 8 mL, 32 eq). The mixture was periodically agitated for 30 minutes, then the solution was drained through the frit. The resin was washed successively three times as follows: for each wash, DMF (7.0 mL) was added to top of the vessel and the resulting mixture was periodically agitated for 90 seconds before the solution was drained through the frit. The reaction was repeated once more. The resin was washed successively five times as follows: for each wash, DMF (7.0 mL) was added to top of the vessel and the resulting mixture was periodically agitated for 90 seconds before the solution was drained through the frit. The resin was washed then successively five times as follows: for each wash, CH₂Cl₂ (5.0 mL) was added to top of the vessel and the resulting mixture was periodically agitated for 90 seconds before the solution was drained through the frit. The resulting resin was placed under a N₂ stream for 10 minutes.

### Global Deprotection Method:

All manipulations were performed manually unless noted. The procedure of “Global Deprotection Method” describes an experiment performed on a 0.05 mmol scale, where the scale is determined by the amount of Rink linker bound to the resin. A “deprotection solution” was prepared by combining in a 100 mL glass vial trifluoroacetic acid (50 mL), DTT (500 mg), and triisopropylsilane (1 mL). The resin was removed from the reaction vessel and transferred to a 50 mL plastic centrifuge tube (VWR-76176-952). To the tube was added the “deprotection solution” (2.0 mL). The mixture was vigorously shaken manually and then on a shaker (200 RPM for 45-60 minutes). To the mixture was added Et₂O (40 mL). The mixture was vigorously mixed upon which a significant amount of a white solid precipitated. The mixture was centrifuged for 3 minutes, then the solution was decanted away from the solids and discarded. The solids were suspended in Et₂O (40 mL) an shaken vigorously again; then the mixture was centrifuged for 3 minutes; and the solution was decanted away from the solids and discarded to afford a mixture of the crude peptide as a white to off-white solid with the resin still in it.

### Cyclization Method:

All manipulations were performed manually unless noted. The procedure of “Cyclization Method” describes an experiment performed on a 0.05 mmol scale, where the scale is determined by the amount of Rink linker bound to the resin that was used to generate the peptide. This scale is not based on a direct determination of the quantity of peptide used in the procedure. The crude peptide solids mixed with the resin were dissolved in 35 ml of DMF, 2 ml of diisopropylethylamine was added. The suspension was then shaken (150 RPM/min.) for 12-18 h. The reaction solution/suspension was concentrated via centrifugal concentration at 35° C. for ˜5 hours, and the residue was then dissolved in 2 ml of DMF and was filtered. The filtrate containing desired product was subjected to reverse-phase HPLC purification to afford the desired cyclic peptide.

### Purification Method A:

The crude material was purified via preparative LC/MS with the following conditions: Column: XBridge C18, 200 mm×30 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 43% B, 43-83% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation.

### Purification Method B:

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 40% B, 40-80% B over 20 minutes, then a 5-minute hold at 100% B; Flow Rate: 20 mL/min; Column Temperature: 25 C. Fraction collection was triggered by MS signals. Fractions containing the desired product were combined and dried via centrifugal evaporation.

### Purity Analysis:

Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

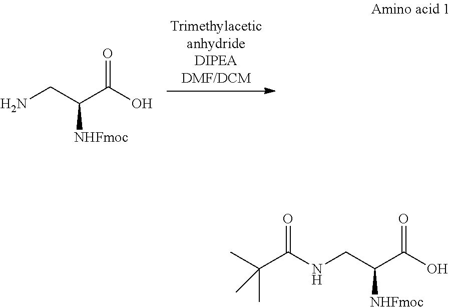

To a suspension of FMOC-DAP-OH (5 g, 15.32 mmol) and diisopropylethylamine (6.69 mL, 38.3 mmol) in DMF (120 mL) and CH₂Cl₂ (150 mL) in a 500 ml of RBF with a magn was added pivalic anhydride (3.42 g, 18.39 mmol) was added to a at 0° C. and the reaction mixture was stirred at rt for 2 h. The reaction mixture was concentrated to remove DCM and the remaining was partitioned between EtOAc/aqueous brine (slightly acidic by adding a few drops of 1.0 M of HCl). The aqueous layer was extracted with EtOAc (5×150 ml). The combined organic layers were washed with brine(5×100 ml), dried over magnesium sulfate, filtered and concentrated under vacuum. The crude product was purified by silica gel chromatography (ISCO Chromatography System; RediSepRf 120 g column; methanol/DCM, Gradient: 0% 50%) to get 6.6 g of desired product which was dissolved in minimum amount of DCM and was diluted with hexane (1000 ml) to precipitate the product as white solid, which was collected by filtering to get desired product/(S)-2-((((9H-fluoren-9-yl)methoxy)carbonyl)amino)-3-pivalamidopropanoic acid (5.5 g, 12.06 mmol, 79% yield) as white solid. ¹H NMR (499 MHz, CHLOROFORM-d) δ 8.05 (s, 1H), 7.78 (br d, J=7.3 Hz, 2H), 7.61 (br t, J=6.3 Hz, 2H), 7.42 (br t, J=7.3 Hz, 2H), 7.37-7.31 (m, 2H), 6.67 (br s, 1H), 6.30 (br s, 1H), 4.41 (br t, J=7.0 Hz, 2H), 4.33 (br s, 1H), 4.28-4.21 (m, 1H), 3.81 (br d, J=13.4 Hz, 1H), 3.63-3.53 (m, 1H), 1.23 (br s, 9H). LCMS: M+1=411

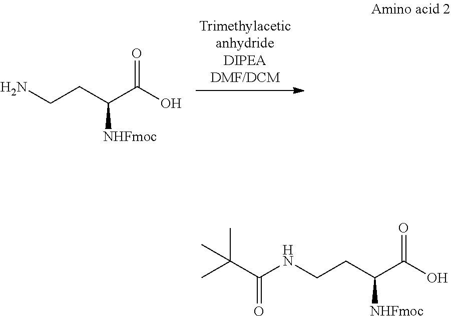

To a suspension of FMOC-DAB-OH (5 g, 14.69 mmol) and DIPEA (6.41 mL, 36.7 mmol) in an DMF (120 mL) CH₂Cl₂ (150 mL) was added pivalic anhydride (3.28 g, 17.63 mmol) at 0° C. and the reaction mixture was stirred at rt for 2 h. The reaction mixture was concentrated to remove DCM and the remaining was partitioned between EtOAc/aqueous brine (slightly acidic by adding a few drops of HCl). The aqueous layer was extracted with EtOAc (4×80 ml). The combined organic layers were washed with brine(4×80 ml), dried over magnesium sulfate, filtered and concentrated under vacuum. The crude product was purified by silica gel chromatography (Biotage Horizon System; RediSepRf 120 g column; methanol/DCM, Gradient: 0%˜50%) to get 7 g of desired product which was dissolved in minimum amount of DCM and was diluted with hexane (1000 ml) to precipitate the product which was collected by filtering to get (S)-2-((((9H-fluoren-9-yl)methoxy)carbonyl)amino)-4-pivalamidobutanoic acid (6 g, 12.72 mmol, 87% yield) as white solid. ¹H NMR (499 MHz, CHLOROFORM-d) δ 7.78 (d, J=7.6 Hz, 2H), 7.61 (t, J=6.6 Hz, 2H), 7.44-7.39 (m, 2H), 7.35-7.30 (m, 2H), 6.69 (br s, 1H), 5.88 (br d, J=7.4 Hz, 1H), 4.42 (br dd, J=6.9, 3.2 Hz, 2H), 4.29 (q, J=7.0 Hz, 1H), 4.22 (t, J=7.0 Hz, 1H), 3.72 (br dd, J=13.5, 5.1 Hz, 1H), 2.99 (s, 1H), 2.13-2.00 (m, 1H), 1.94-1.83 (m, 1H), 1.28-1.24 (m, 9H). LCMS: M+1=425.05.

The Examples shown in Table 2 were prepared using the methods described above.

TABLE 2

| Example | |
|---|---|
| Number | Structure |
| 20010 | 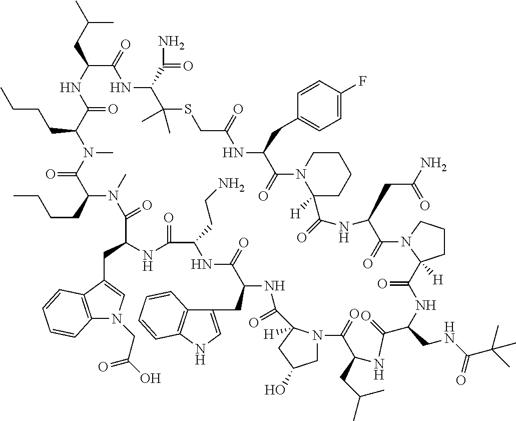 [SMILES: CCCC[C@H]1C(=O)N(C)[C@@H](CCCC)C(=O)N[C@@H](CC(C)C)C(=O)N[C@H](C(N)=O)C(C)(C)SCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20020 | 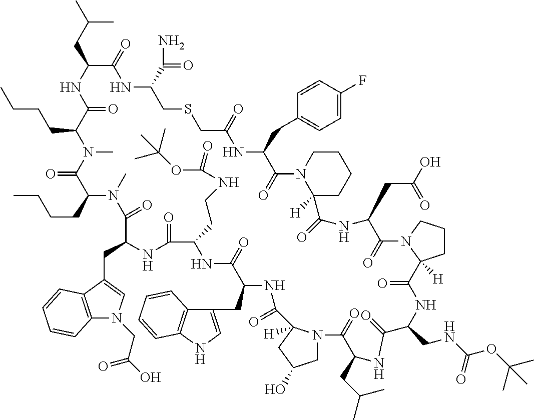 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)OC(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)OC(C)(C)C)C(=O)N[C@@H](Cc2cn(CC(=O)O)c3ccccc23)C(=O)N1C] |
| 20030 | 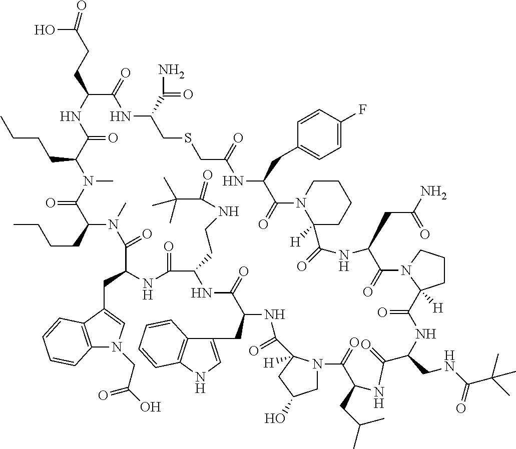 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20040 | 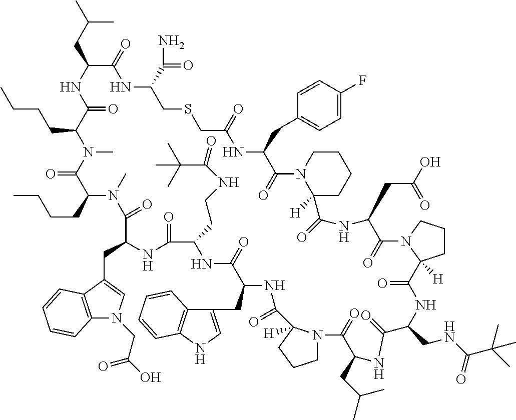 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20050 | 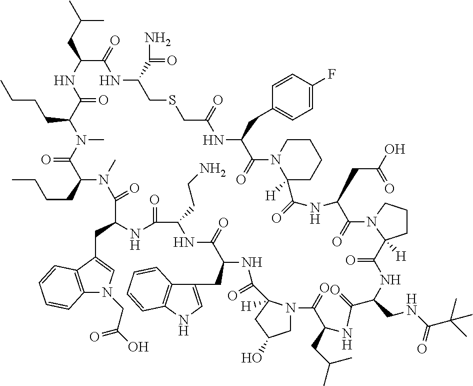 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20060 | 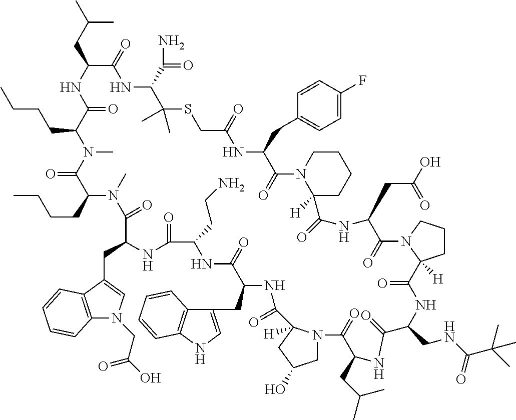 [SMILES: CCCC[C@H]1C(=O)N(C)[C@@H](CCCC)C(=O)N[C@@H](CC(C)C)C(=O)N[C@H](C(N)=O)C(C)(C)SCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20070 | 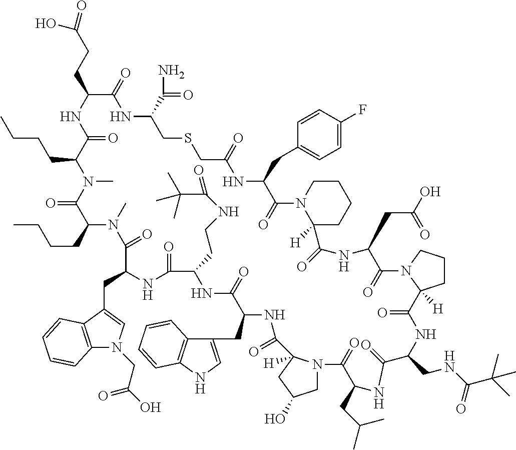 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20080 | 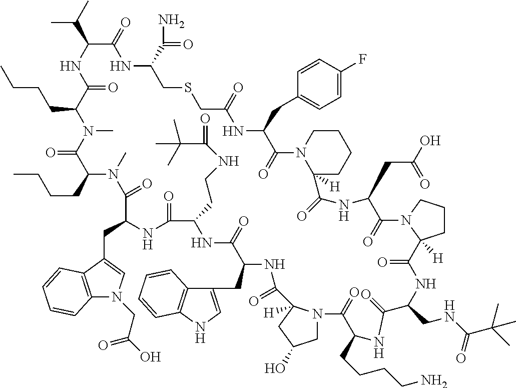 [SMILES: CCCC[C@H]1C(=O)N(C)[C@@H](CCCC)C(=O)N[C@@H](C(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CCCCN)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20090 | 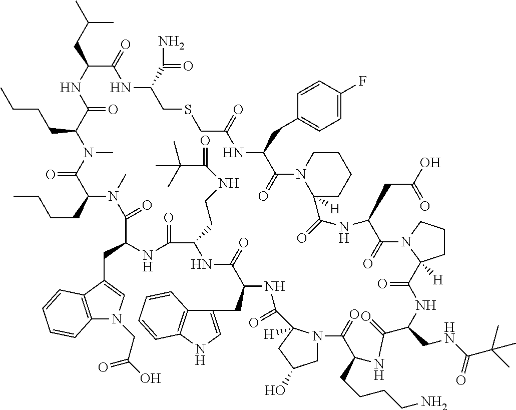 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CCCCN)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20110 | 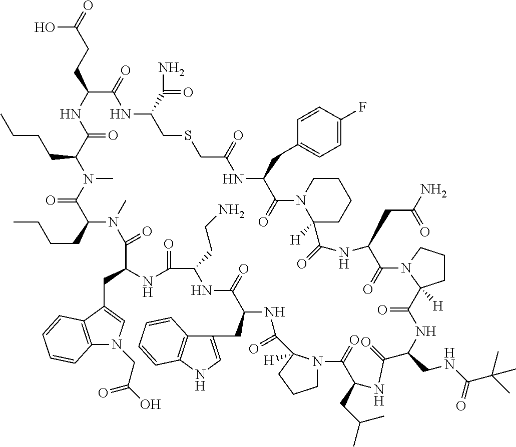 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2CCC[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20120 | 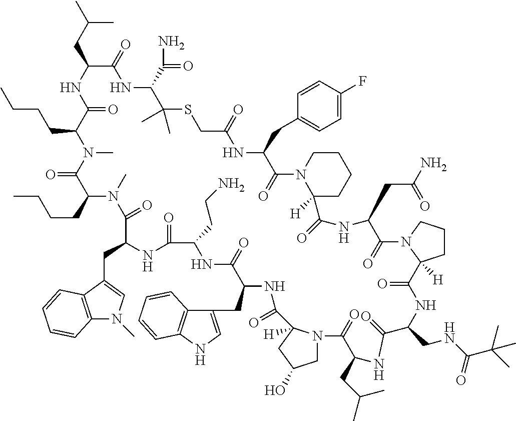 [SMILES: CCCC[C@H]1C(=O)N(C)[C@@H](CCCC)C(=O)N[C@@H](CC(C)C)C(=O)N[C@H](C(N)=O)C(C)(C)SCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20130 | 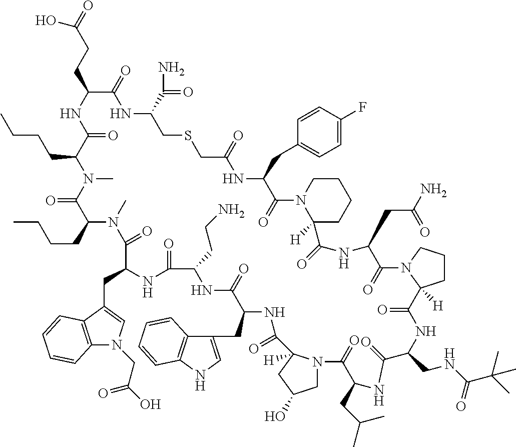 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20150 | 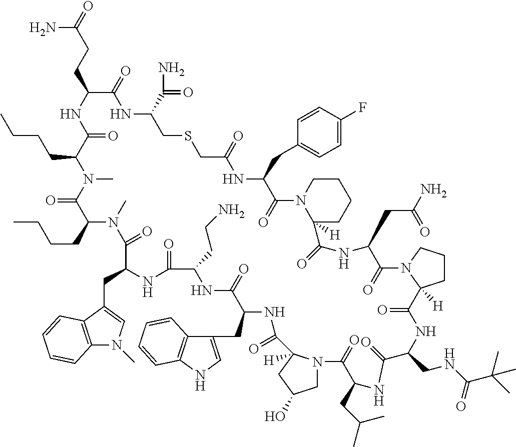 [SMILES: CCCC[C@H]1C(=O)N(C)[C@@H](CCCC)C(=O)N[C@@H](CCC(N)=O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20160 | 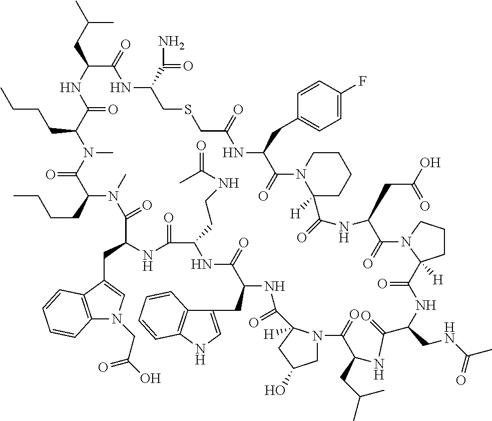 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(C)=O)C(=O)N[C@@H](CC(C)C)C(=O)N2C[C@H](O)C[C@H]2C(=O)N[C@@H](Cc2c[nH]c3ccccc23)C(=O)N[C@@H](CCNC(C)=O)C(=O)N[C@@H](Cc2cn(CC(=O)O)c3ccccc23)C(=O)N1C] |
| 20170 | 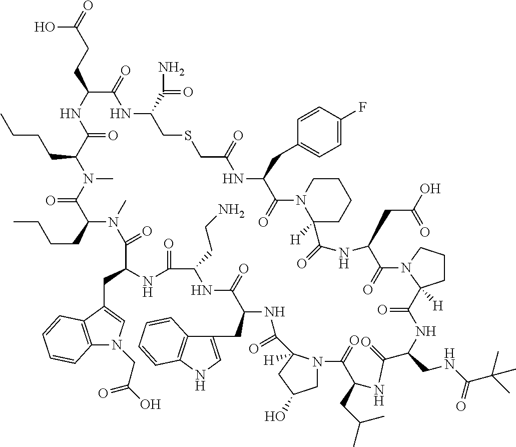 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20180 | 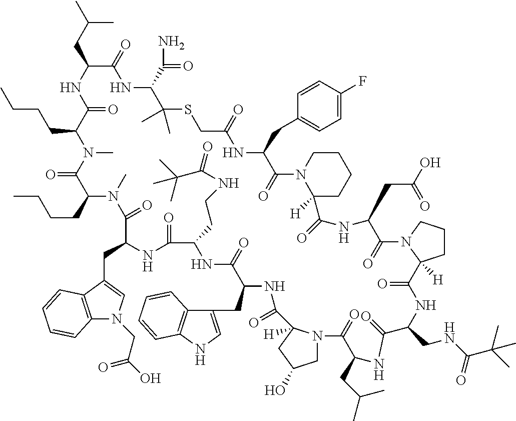 [SMILES: CCCC[C@H]1C(=O)N(C)[C@@H](CCCC)C(=O)N[C@@H](CC(C)C)C(=O)N[C@H](C(N)=O)C(C)(C)SCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20190 | 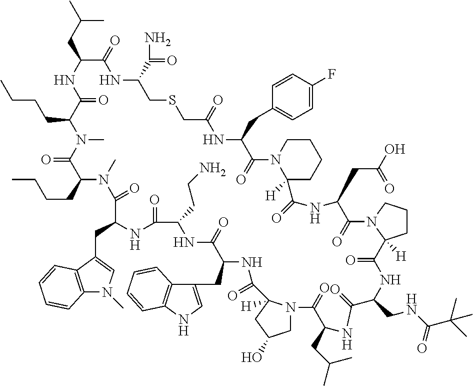 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20200 | 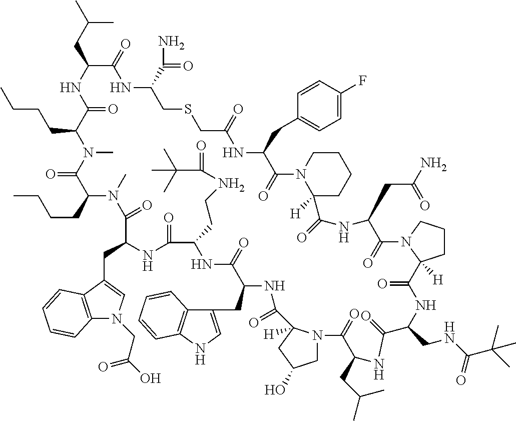 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20210 | 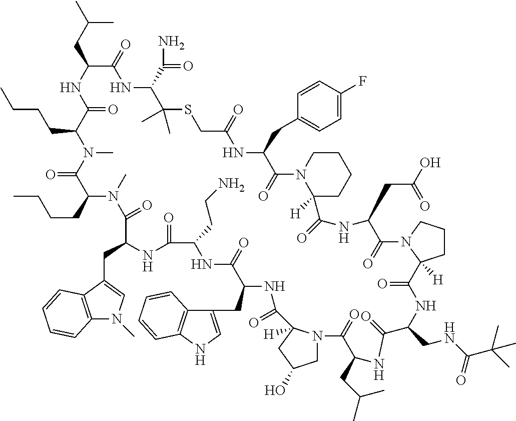 [SMILES: CCCC[C@H]1C(=O)N(C)[C@@H](CCCC)C(=O)N[C@@H](CC(C)C)C(=O)N[C@H](C(N)=O)C(C)(C)SCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20220 | 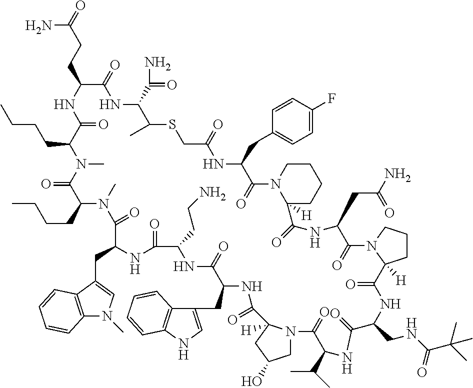 [SMILES: CCCC[C@H]1C(=O)N(C)[C@@H](CCCC)C(=O)N[C@@H](CCC(N)=O)C(=O)N[C@H](C(N)=O)C(C)SCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](C(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20230 | 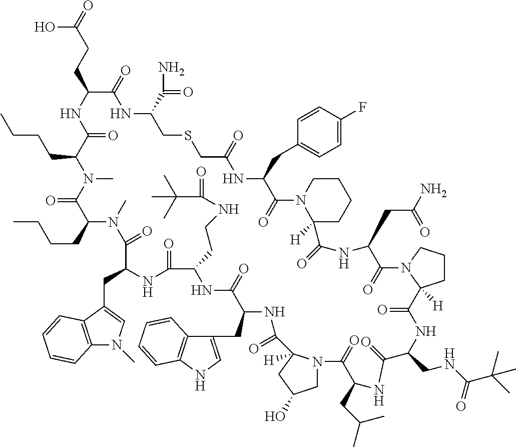 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(C)c3ccccc23)C(=O)N1C] |
| 20240 | 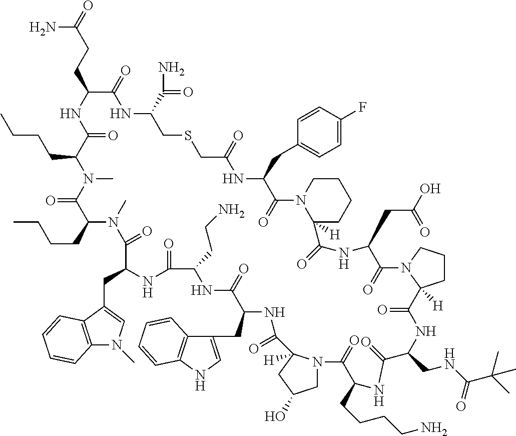 [SMILES: CCCC[C@H]1C(=O)N(C)[C@@H](CCCC)C(=O)N[C@@H](CCC(N)=O)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CCCCN)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20250 | 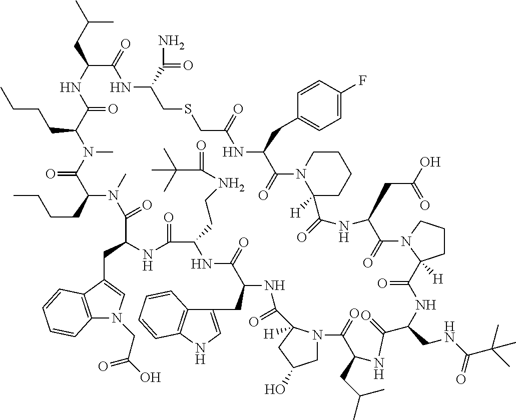 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(CC(=O)O)c3ccccc23)C(=O)N1C] |
| 20260 | 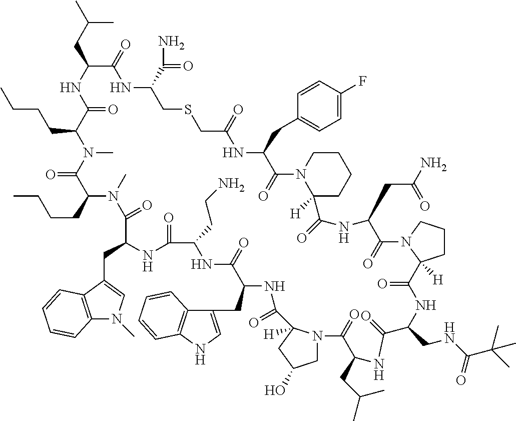 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20270 | 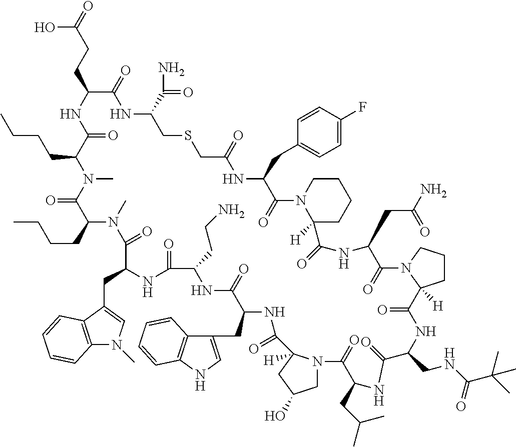 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(C)c3ccccc23)C(=O)N1C] |
| 20280 | 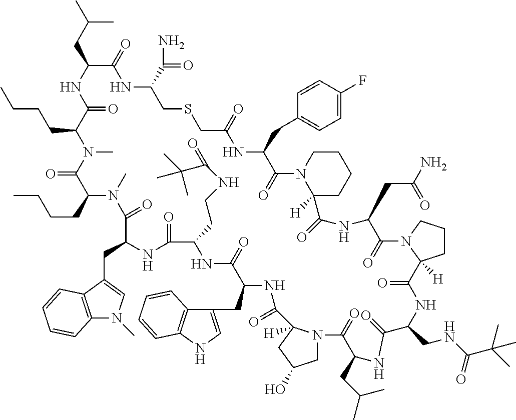 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(C)c3ccccc23)C(=O)N1C] |
| 20290 | 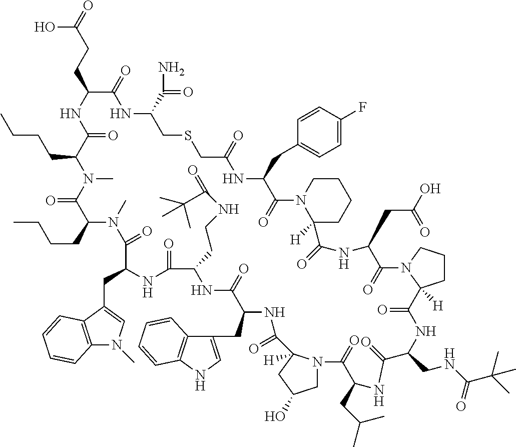 [SMILES: CCCC[C@H]1C(=O)N(C)[C@@H](CCCC)C(=O)N[C@@H](CCC(=O)O)C(=O)N[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCNC(=O)C(C)(C)C)C(=O)N[C@@H](Cc2cn(C)c3ccccc23)C(=O)N1C] |
| 20300 | 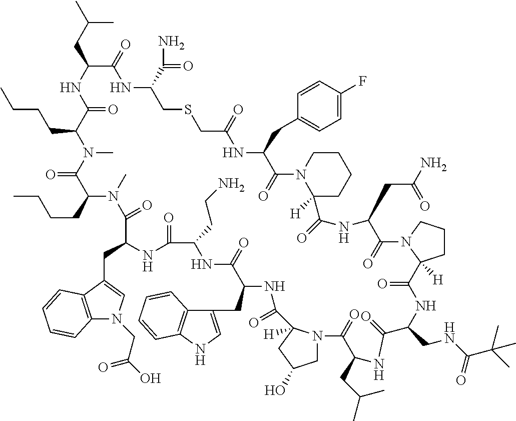 [SMILES: CCCC[C@H]1C(=O)N(C)[C@@H](CCCC)C(=O)N[C@@H](CC(C)C)C(=O)N[C@H](C(N)=O)CSCC(=O)N[C@@H](Cc2ccc(F)cc2)C(=O)N2CCCC[C@H]2C(=O)N[C@@H](CC(N)=O)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C] |
| 20310 | 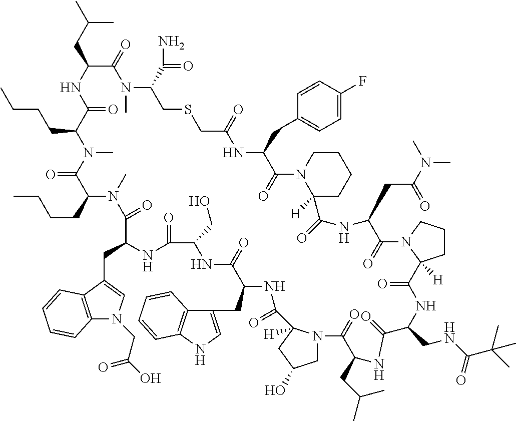 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(=O)N(C)C)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C] |

### General Procedures:

All manipulations were performed under automation on a Prelude peptide synthesizer (Protein Technologies). All procedures unless noted were performed in a 40 ml reaction vessel fitted with a bottom frit. The vessel connects to a the Prelude peptide synthesizer through both the bottom and the top of the vessel. DMF and DCM can be added through the top of the vessel, which washes down the sides of the tube equally. The remaining reagents are added through the bottom of the tube and pass up through the frit to contact the resin. All solutions are removed through the bottom of the tube. “Periodic agitation” describes a brief pulse of N2 gas through the bottom frit; the pulse lasts approximately 5 seconds and occurs every 30 seconds. 0.4 M of Chloroacetyl anhydride solutions in DMF were used within 5 days of preparation. Amino acid solutions were generally not used beyond three weeks from preparation. HATU solutions were used within 5 days of preparation. DMF=dimethylformamide; HATU=1-[Bis(dimethylamino)methylene]-1H-1,2,3-triazolo[4,5-b]pyridinium 3-oxide hexafluorophosphate; DIPEA=diisopropylethylamine; Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.53 mmol/g loading. Common amino acids used are listed below with side-chain protecting groups indicated inside parenthesis.

Fmoc-Ala-OH; Fmoc-Arg(Pbf)-OH; Fmoc-Asn(Trt)-OH; Fmoc-Asp(OtBu)-OH; Fmoc-Bzt-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc[N-Me]Nle-OH; Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH

The procedures of “Prelude Method A” describe an experiment performed on a 0.2 mmol scale, where the scale is determined by the amount of Rink linker bound to the resin. This scale corresponds to approximately 378 mg of the Rink-Merrifield resin described above. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus used the “Double-coupling procedure” described below. Coupling of chloroacetylchloride to the N-terminus of the peptide is described by the “Chloroacetyl chloride coupling procedure” detailed below.

### Resin-Swelling Procedure:

To a 40 mL polypropylene solid-phase reaction vessel was added Merrifield:Rink resin (378 mg, 0.200 mmol). The resin was washed (swelled) three times as follows: to the reaction vessel was added DMF (10.0 mL), upon which the mixture was periodically agitated for 10 minutes before the solvent was drained through the frit. The swelling was repeated one more time and the DMF was remove from the bottom of the vessel.

### Single-Coupling Procedure:

To the reaction vessel containing resin from the previous step was added piperidine:DMF (20:80 v/v, 6.0 mL). The mixture was periodically agitated for 5 minutes and then the solution was drained through the frit. To the reaction vessel was added piperidine:DMF (20:80 v/v, 6.0 mL). The mixture was periodically agitated for 5 minutes and then the solution was drained through the frit. The resin was washed successively six times as follows: for each wash, DMF (7.0 mL) was added through the top of the vessel and the resulting mixture was periodically agitated for 1 min. before the solution was drained through the frit. To the reaction vessel was added the amino acid (0.2M in DMF, 5.0 mL, 5 eq), then HATU (0.4M in DMF, 2.5 mL, 5 eq), and finally NMM (N-methylmorpholine, 0.8M in DMF, 0.25 mL, 10 eq). The mixture was periodically agitated for 2 hours, then the reaction solution was drained through the frit. The resin was washed successively five times as follows: for each wash, DMF (7.0 mL) was added through the top of the vessel and the resulting mixture was periodically agitated for 1 min. before the solution was drained through the frit. The resulting resin was used directly in the next step.

### Double-Coupling Procedure Used for Secondary Amino Acids:

After the first single-coupling procedure was finished it was repeated once more to make sure the coupling is completed.

### Chloroacetyl Chloride Coupling Procedure:

To the reaction vessel containing the resin from the previous step was added piperidine:DMF (20:80 v/v, 6.0 mL). The mixture was periodically agitated for 5 minutes and then the solution was drained through the frit. To the reaction vessel was added piperidine:DMF (20:80 v/v, 6.0 mL). The mixture was periodically agitated for 5 minutes and then the solution was drained through the frit. The resin was washed successively six times as follows: for each wash, DMF (7.0 mL) was added through the top of the vessel and the resulting mixture was periodically agitated for 1 min. before the solution was drained through the frit. To the reaction vessel was added chloroacetyl chloride (0.4M in DMF, 8 mL, 16 eq) and then NMM (N-methylmorpholine, 0.8M in DMF, 8 mL, 32 eq). The mixture was periodically agitated for 30 minutes, then the solution was drained through the frit. The resin was washed successively three times as follows: for each wash, DMF (7.0 mL) was added to top of the vessel and the resulting mixture was periodically agitated for 90 seconds before the solution was drained through the frit. The reaction was repeated once more. The resin was washed successively five times as follows: for each wash, DMF (7.0 mL) was added to top of the vessel and the resulting mixture was periodically agitated for 90 seconds before the solution was drained through the frit. The resin was washed then successively five times as follows: for each wash, CH₂Cl₂ (5.0 mL) was added to top of the vessel and the resulting mixture was periodically agitated for 90 seconds before the solution was drained through the frit. The resulting resin was placed under a N₂ stream for 10 minutes.

### Global Deprotection Method:

All manipulations were performed manually unless noted. The procedure of “Global Deprotection Method” describes an experiment performed on a 0.05 mmol scale, where the scale is determined by the amount of Rink linker bound to the resin. A “deprotection solution” was prepared by combining in a 100 mL glass vial trifluoroacetic acid (50 mL), DTT (500 mg), and triisopropylsilane (1 mL). The resin was removed from the reaction vessel and transferred to a 50 mL plastic centrifuge tube. To the tube was added the “deprotection solution” (2.0 mL). The mixture was vigorously shaken manually and then on a shaker (200 RPM for 45-60 minutes). To the mixture was added Et₂O (40 mL). The mixture was vigorously mixed upon which a significant amount of a white solid precipitated. The mixture was centrifuged for 3 minutes, then the solution was decanted away from the solids and discarded. The solids were suspended in Et₂O (40 mL) an shaken vigorously again; then the mixture was centrifuged for 3 minutes; and the solution was decanted away from the solids and discarded to afford a mixture of the crude peptide as a white to off-white solid with the resin still in it.

### Cyclization Method:

All manipulations were performed manually unless noted. The procedure of “Cyclization Method” describes an experiment performed on a 0.05 mmol scale, where the scale is determined by the amount of Rink linker bound to the resin that was used to generate the peptide. This scale is not based on a direct determination of the quantity of peptide used in the procedure. The crude peptide solids mixed with the resin were dissolved in 35 ml of DMF, 2 ml of diisopropylethylamine was added. The suspension was then shaken (150 RPM/min.) for 12-18 h. The reaction solution/suspension was concentrated via centrifugal concentration at 35° C. for ˜5 hours, and the residue was then dissolved in 2 ml of DMF and was filtered. The filtrate containing desired product was subjected to reverse-phase HPLC purification to afford the desired cyclic peptide.

### Preparation of Intermediate 1

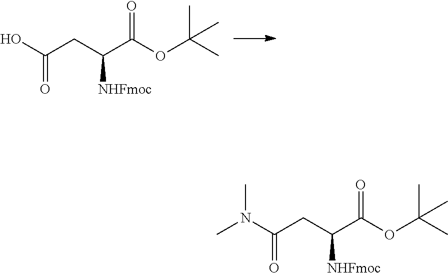

To a solution of Fmoc-Asp(OH)—OtBu (12 g, 29.2 mmol), dimethylamine (2 M in THF) (20.42 mL, 40.8 mmol) and DIPEA (10.19 mL, 58.3 mmol) in DCM (100 mL) was added HATU (14.42 g, 37.9 mmol). The resulting mixture was stirred at rt for 2 h. The reaction mixture was concentrated to remove most of the solvent and the remaining was partitioned between EtOAc/aqueous sodium bicarbonate. The aqueous layer was extracted with EtOAc (3×100 ml). The combined organic layers were washed with aqueous sodium bicarbonate (4×100 ml) and brine(3×100 ml), dried over magnesium sulfate, filtered and concentrated under vacuum to get tert-butyl N2-(((9H-fluoren-9-yl)methoxy)carbonyl)-N4,N4-dimethyl-L-asparaginate (13 g, 26.7 mmol, 91% yield) as light-yellow foamy solid.

¹H NMR (499 MHz, CHLOROFORM-d) δ 7.78 (d, J=7.5 Hz, 2H), 7.64 (dd, J=7.4, 3.8 Hz, 2H), 7.42 (t, J=7.5 Hz, 2H), 7.35-7.30 (m, 2H), 6.15 (br d, J=9.1 Hz, 1H), 4.59-4.51 (m, 1H), 4.45 (dd, J=10.2, 7.0 Hz, 1H), 4.32-4.28 (m, 1H), 4.26 (d, J=7.2 Hz, 1H), 3.14 (dd, J=16.6, 4.0 Hz, 1H), 3.02 (s, 3H), 2.97 (s, 3H), 2.82 (s, 1H), 1.49 (s, 9H)

### Preparation of Intermediate 2

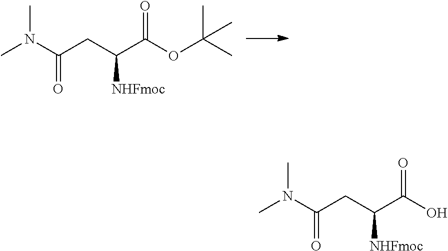

To a solution of tert-butyl N2-(((9H-fluoren-9-yl)methoxy)carbonyl)-N4,N4-dimethyl-L-asparaginate (13 g, 29.6 mmol) and TRIISOPROPYLSILANE (5.63 g, 35.6 mmol) in CH₂Cl₂ (200 mL) was added TFA (114 mL, 1482 mmol) at 0° C. The resulting mixture was stirred at rt under nitrogen for 2 h. The reaction mixture was concentrated. The residue was partitioned between EtOAc/brine. The pH of the aqueous layer was neutralized with 1.0 M sodium hydroxide to pH=˜6. The combined organic layers were washed with brine (3×100 ml) and was then dried over magnesium sulfate, filtered and concentrated under vacuum. The crude product was purified by silica gel chromatography (ISCO System; RediSepRf 240 g column; methanol/DCM, Gradient: 0%˜30%) to get N2-(((9H-fluoren-9-yl)methoxy)carbonyl)-N4,N4-dimethyl-L-asparagine (8 g, 24.84 mmol, 65% yield) as white solid. ¹H NMR (499 MHz) δ 7.74 (d, J=7.4 Hz, 1H), 7.74 (d, J=7.4 Hz, 1H), 7.56 (d, J=7.5 Hz, 1H), 7.56 (d, J=7.5 Hz, 1H), 7.38 (dd, J=7.5, 7.4 Hz, 1H), 7.38 (dd, J=7.5, 7.4 Hz, 1H), 7.31 (t, J=7.5 Hz, 1H), 7.31 (t, J=7.5 Hz, 1H), 4.62 (ddd, J=9.3, 8.3, 4.3 Hz, 1H), 4.47 (d, J=6.7 Hz, 2H), 4.07 (t, J=6.7 Hz, 1H), 3.00 (s, 3H), 2.94 (s, 3H), 2.87 (dd, J=15.0, 4.3 Hz, 1H), 2.78 (dd, J=15.0, 9.3 Hz, 1H). LCMS: M+1=383

### Preparation of Example 10001

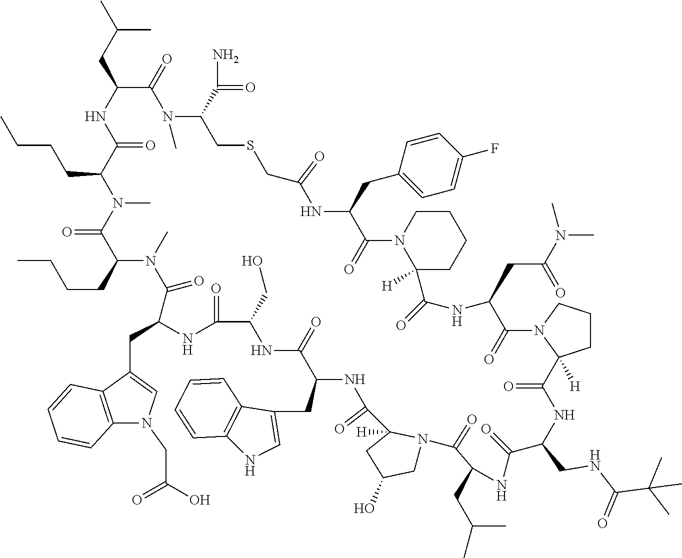 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(=O)N(C)C)C(=O)N2CCC[C@H]2C(=O)N[C@@H](CNC(=O)C(C)(C)C)C(=O)N[C@@H](CC(C)C)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

Example 10001 was prepared following the general procedures described above. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 35% B, 35-75% B over 20 minutes, then a 4-minute hold at 100% B; Flow Rate: 20 mL/min; Column Temperature: 25 C. Fraction collection was triggered by UV signals. Fractions containing the desired product were combined and dried via centrifugal evaporation. The estimated purity of the product by LCMS analysis was 95%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 95.3%; Observed Mass: 986.16; Retention Time: 2.07 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 95.1%; Observed Mass: 987; Retention Time: 2.34 min.

### Preparation of Example 10002

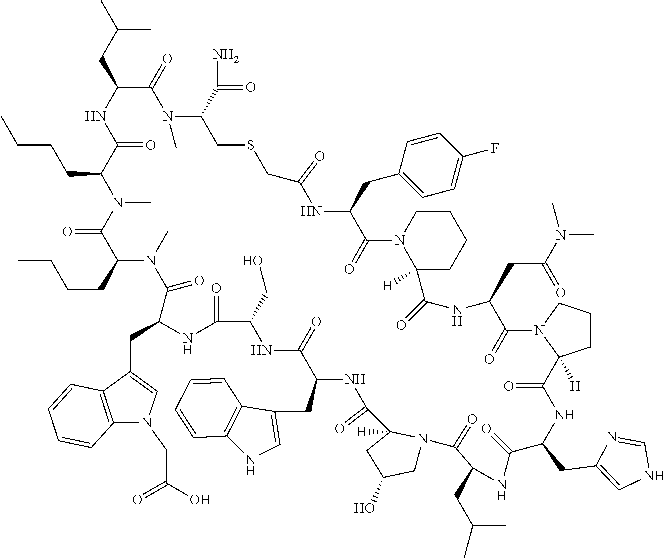 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(=O)N(C)C)C(=O)N2CCC[C@H]2C(=O)N[C@@H](Cc2c[nH]cn2)C(=O)N[C@@H](CC(C)C)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

Example 10002 was prepared following the general procedures described above. The crude product was purified via preparative HPLC described for example 1. The purity of the product was 94%. M+H=1938.

All examples exemplified below were prepared by following similar procedure to the “General Procedures” above on the synthesizer

### Preparation of Example 10003

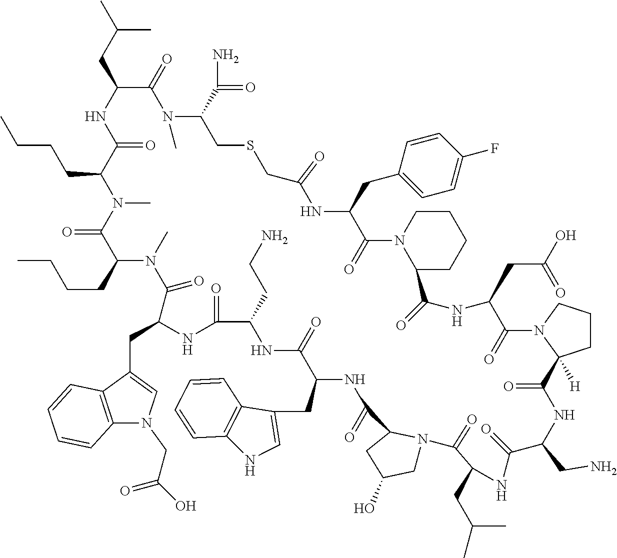 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

The crude material was purified via preparative LC/MS with the following conditions: Column: Waters CSH Fluoro Phenyl, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 20% B, 20-60% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The product estimated purity by LCMS analysis was 92%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 92.6%; Observed Mass: 1886.87, 1888.1; Retention Time: 1.84, 1.98 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 92.1%; Observed Mass: 1887.12; Retention Time: 2.06 min.

### Preparation of Example 10004

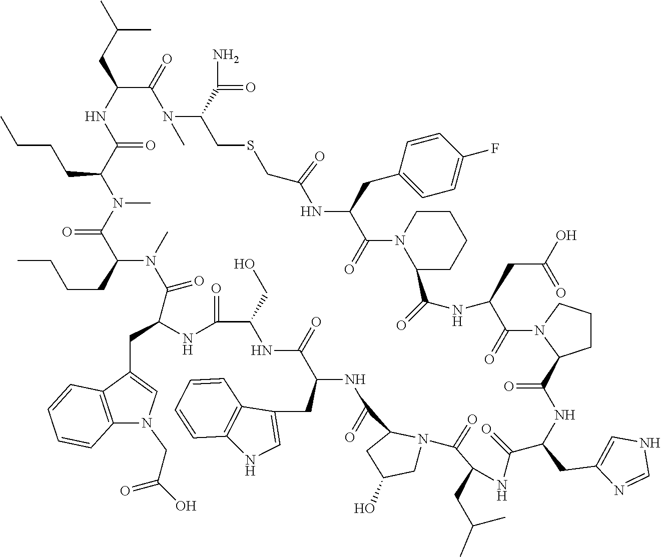 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(=O)O)C(=O)N2CCC[C@H]2C(=O)N[C@@H](Cc2c[nH]cn2)C(=O)N[C@@H](CC(C)C)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 32% B, 32-72% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The product estimated purity by LCMS analysis was 99%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 1912.1; Retention Time: 1.71 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 98.9%; Observed Mass: 1911.16; Retention Time: 2.01 min.

### Preparation of Example 10005

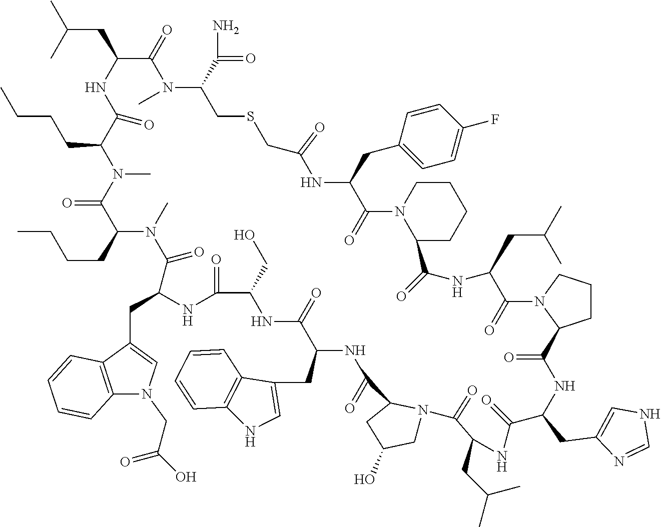 [SMILES: CCCC[C@H]1C(=O)N(C)[C@@H](CCCC)C(=O)N[C@@H](CC(C)C)C(=O)N(C)[C@H](C(N)=O)CSCC(=O)N[C@@H](Cc2ccc(F)cc2)C(=O)N2CCCC[C@H]2C(=O)N[C@@H](CC(C)C)C(=O)N2CCC[C@H]2C(=O)N[C@@H](Cc2c[nH]cn2)C(=O)N[C@@H](CC(C)C)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

The crude material was purified via preparative LC/MS with the following conditions: Column: Waters CSH Fluoro Phenyl, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 31% B, 31-71% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 32% B, 32-72% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 35% B, 35-75% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The product estimated purity by LCMS analysis was 97%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 955.16; Retention Time: 2.04 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 96.7%; Observed Mass: 955.3; Retention Time: 2.09 min. Analysis condition B: Retention time=2.84 min; ESI-MS(+) m/z 964.1 (M+2H).

### Biological Activity

The ability of the compounds of formula (I) to bind to PD-L1 was investigated using a PD-1/PD-L1 Homogenous Time-Resolved Fluorescence (HTRF) binding assay.

### Homogenous Time-Resolved Fluorescence (HTRF) Binding Assay

The interaction of PD-1 and PD-L1 can be assessed using soluble, purified preparations of the extracellular domains of the two proteins. The PD-1 and PD-L1 protein extracellular domains were expressed as fusion proteins with detection tags, for PD-1, the tag was the Fc portion of Immunoglobulin (PD-1-Ig) and for PD-L1 it was the 6 histidine motif (PD-L1-His). All binding studies were performed in an HTRF assay buffer consisting of dPBS supplemented with 0.1% (with) bovine serum albumin and 0.05% (v/v) Tween-20. For the h/PD-L1-His binding assay, inhibitors were pre-incubated with PD-L1-His (10 nM final) for 15 m in 4 μl of assay buffer, followed by addition of PD-1-Ig (20 nM final) in 1 μl of assay buffer and further incubation for 15 m. HTRF detection was achieved using europium cryptate-labeled anti-Ig (1 nM final) and allophycocyanin (APC) labeled anti-His (20 nM final). Antibodies were diluted in HTRF detection buffer and 5 μl was dispensed on top of the binding reaction. The reaction mixture was allowed to equilibrate for 30 minutes and the resulting signal (665 nm/620 nm ratio) was obtained using an EnVision fluorometer. Additional binding assays were established between the human proteins PD-1-Ig/PD-L2-His (20 & 5 nM, respectively) and CD80-His/PD-L1-Ig (100 & 10 nM, respectively).

Recombinant Proteins: Human PD-1 (25-167) with a C-terminal human Fc domain of immunoglobulin G (Ig) epitope tag [hPD-1 (25-167)-3S-IG] and human PD-L1 (18-239) with a C-terminal His epitope tag [hPD-L1(18-239)-TVMV-His] were expressed in HEK293T cells and purified sequentially by Protein A affinity chromatography and size exclusion chromatography. Human PD-L2-His and CD80-His was obtained through commercial sources.

```
Sequence of recombinant human PD-1-Ig
hPD1(25-167)-3S-IG
(SEQ ID NO: 1)
    1 LDSPDRPWNP PTFSPALLVV TEGDNATFTC
        SFSNTSESFV LNWYRMSPSN
  51 QTDKLAAFPE DRSQPGQDCR FRVTQLPNGR
        DFHMSVVRAR RNDSGTYLCG
101 AISLAPKAQI KESLRAELRV TERRAEVPTA
        HPSPSPRPAG QFQGSPGGGG
151 GREPKSSDKT HTSPPSPAPE LLGGSSVFLF
        PPKPKDTLMI SRTPKVTCVV
201 VDVSHEDPEV KENICIVDGVE VENAKTKPRE
        EQYNSTYRVV SVLTVLHQDW
251 LNGKEYKCKV SNKALPAPIE KTISKAKGQF
        REFQVYTLPP SRDELTKNQV
301 SLTCLVKGFY PSDIAVEWES NGQPENNYKT
        TFFVLDSDGS FFLYSKLTVD
351 KSRWQQGNVF SCSVMHEALH NHYTQKSISL
        SPGK
Sequence of recombinant human PD-L1-His
hPDL1(18-239)-TVMV-His
(SEQ ID NO: 2)
    1 AFTVTVPKDL YVVEYGSNMT IECKFPVEKQ
        LDLAALIVYW EMEDKNIIQF
  51 VHGEEDLKVQ HSSYRQRARL LKDQLSLGNA
        ALQITDVKLQ DAGVYRCMIS
101 YGGADYKRIT VKVNAPYNKI NQRILVVDPV
        TSEHELTCQA EGYPKAEVIW
151 TSSDHQVLSG KTTTTNSKRE EKLFNVTSTL
        RINTTTNEIF YCTFRRLDPE
201 ENHTAELVIP ELPLAHPPNE RTGSSETVRF
        QGNHHRHH
```

Table 3 lists the IC₅₀ values for representative examples of this disclosure measured in the PD-1/PD-L1 Homogenous Time-Resolved Fluorescence (HTRF) binding assay.

TABLE 3

| Example Number | HTRF IC₅₀ (nM) |
|---|---|
| 1003 | 37 |
| 1004 | >10000 |
| 1005 | >10000 |
| 1006 | 22 |
| 1007 | 3.3 |
| 1008 | N/A |
| 1009 | 5.5 |
| 1010 | 3.9 |
| 1012 | 19 |
| 1013 | 1100 |
| 1014 | 6.1 |
| 1015 | >10000 |
| 1016 | 12 |
| 1017 | 5.9 |
| 1018 | 2.9 |
| 1019 | 1.2 |
| 1020 | 7.1 |
| 1021 | 52 |
| 1022 | 15 |
| 1023 | 6.8 |
| 1024 | 4.7 |
| 1025 | 6.0 |
| 1026 | 4.5 |
| 1027 | 2.0 |
| 1029 | 4.9 |
| 1030 | 4.0 |
| 1031 | 4.7 |
| 1032 | 0.74 |
| 1033 | 7.6 |
| 1035 | 8.3 |
| 1036 | 1.4 |
| 1037 | 5.1 |
| 1039 | 1.5 |
| 1040 | 3.0 |
| 1041 | 4.4 |
| 1042 | 11.4 |
| 1043 | 3.0 |
| 1044 | 2.6 |
| 1045 | 4.6 |
| 1046 | 2.6 |
| 1048 | 1.1 |
| 2001 | 3.4 |
| 2002 | 1.2 |
| 2003 | 24 |
| 20010 | 4.9 |
| 20020 | 6.9 |
| 20030 | 7.8 |
| 20040 | 8.3 |
| 20050 | 10.4 |
| 20060 | 10.4 |
| 20070 | 10.5 |
| 20080 | 10.7 |
| 20090 | 11.1 |
| 20110 | 11.7 |
| 20120 | 11.8 |
| 20130 | 11.8 |
| 20150 | 12.5 |
| 20170 | 14.0 |
| 20180 | 14.1 |
| 20190 | 14.2 |
| 20200 | 15.8 |
| 20210 | 16.4 |
| 20220 | 16.6 |
| 20230 | 16.6 |
| 20240 | 18.4 |
| 20250 | 22.9 |
| 20260 | 28.9 |
| 20270 | 43.2 |
| 20280 | 50.3 |
| 20290 | 70.1 |
| 20300 | 38.2 |
| 20310 | 18.0 |
| 10001 | 18 |
| 10002 | 52 |
| 10003 | 12 |
| 10004 | 11 |

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A compound of formula (I) 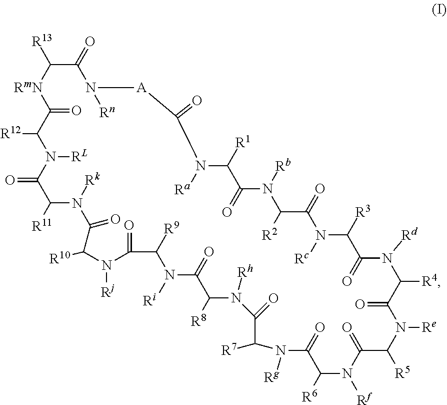
or a pharmaceutically acceptable salt thereof, wherein:
A is selected from a bond, 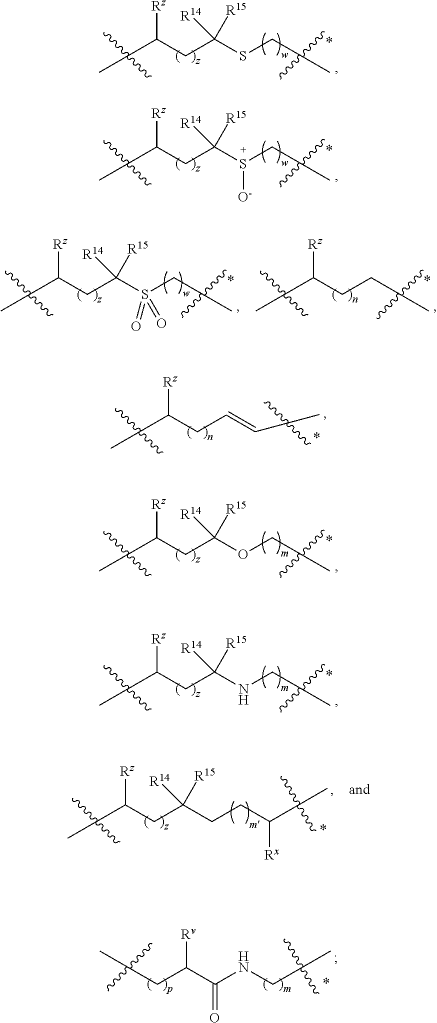
wherein: [symbol]
denotes the point of attachment to the carbonyl group and [symbol] denotes the point of attachment to the nitrogen atom;
z is 0, 1, or 2;
w is 1 or 2;
n is 0 or 1;
m is 1 or 2;
m′ is 0 or 1;
p is 0, 1, or 2;
R^{x} is hydrogen, amino, hydroxy, or methyl;
R¹⁴ and R¹⁵ are independently hydrogen or methyl; and
R^{z} is hydrogen or —C(O)NHR¹⁶; wherein R¹⁶ is hydrogen, —CHR¹⁷C(O)NH₂, —CHR¹⁷C(O)NHCHR¹⁸C(O)NH₂, or —CHR¹⁷C(O)NHCHR¹⁸C(O)NHCH₂C(O)NH₂; wherein R¹⁷ is hydrogen or —CH₂OH and wherein R¹⁸ is hydrogen or methyl;
R^{v} is hydrogen or a natural amino acid side chain;
R^{c}, R^{f}, R^{h}, Rⁱ, and R^{m} are hydrogen;
Rⁿ is hydrogen or methyl or, when p is 0, R^{v} and Rⁿ, together with the atoms to which they are attached, can form a pyrrolidine ring;
R^{a}, R^{e}, and R^{j} are each independently hydrogen or methyl;
R⁵ is —(CH₂)_{q}NR⁵⁰R⁵¹, a natural amino acid side chain, or an unnatural amino acid side chain;
R⁹ is —(CH₂)_{q′}NR⁵⁰R^{51′}, a natural amino acid side chain, or an unnatural amino acid side chain;
provided that at least one of R⁵ and R⁹ is other than a natural amino acid side chain or an unnatural amino acid side chain;
q and q′ are each independently 1 or 2;
R⁵⁰, R⁵¹, R^{50′}, and R^{51′} are each independently hydrogen, C₁-C₁₃alkoxycarbonyl, C₄-C₁₃alkylcarbonyl, C₁-C₁₃alkylsulfanylcarbonyl, C₁-C₁₃haloalkoxycarbonyl, C₁-C₁₃haloalkylcarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹;
R⁷⁰ and R⁷¹ are independently hydrogen, C₁-C₁₃alkoxy, C₁-C₁₃alkyl, C₁-C₁₃alkylcarbonyl, C₃-C₁₄cycloalkyl, or phenylC₁-C₃alkyl wherein the phenyl part of the phenylC₁-C₃alkyl is optionally substituted with one, two, or three groups wherein each group is independently C₁-C₃alkoxy, C₁-C₃alkyl, or C₁-C₃alkylcarbonyl, and wherein the phenyl part of the phenylC₁-C₃alkyl is optionally fused to a dioxolanyl ring;
R⁹⁰ and R⁹¹ are independently hydrogen or C₁-C₆alkyl;
provided that when R⁵ is —(CH₂)_{q}NR⁵⁰R⁵¹ and R⁹ is an amino acid side chain or an unnatural amino acid side chain, at least one of R⁵⁰ and R⁵¹ is other than hydrogen;
provided that when R⁹ is —(CH₂)_{q′}NR^{50′}R^{51′} and R⁵ is an amino acid side chain or an unnatural amino acid side chain, at least one of R^{50′} and R^{51′} is other than hydrogen; and
provided that when R⁵ is —(CH₂)_{q′}NR^{50′}R^{51′} and R⁹ is —(CH₂)_{q′}NR^{50′}R^{51′}; at least one of R⁵⁰, R⁵¹, R^{50′} and R^{51′} is other than hydrogen;
R¹, R², R³, R⁴, R⁶, R⁷, R⁸, R¹⁰, R¹¹, R¹², and R¹³ are each independently a natural amino acid side chain or an unnatural amino acid side chain; or
R², R⁴, R⁶, R⁷, R⁸, R¹⁰, R¹¹, R¹², and R¹³ can each independently form a ring with the corresponding vicinal R group as described below;
R^{b} is methyl or R^{b} and R², together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxy;
R^{d} is hydrogen or methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;
R^{g} is hydrogen or methyl, or R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;
R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy;
R^{L} is methyl or R^{L} and R¹², together with the atoms to which they are attached, form an azetidine or pyrrolidine ring, wherein each ring is optionally substituted with one to four groups wherein each group is independently amino, cyano, methyl, halo, or hydroxy;
provided that the compound of formula (I) contains at least one carbon on the backbone of the ring that has four substituents other than hydrogen and is not an alpha-methyl-substituted ring.

2. A compound of claim 1, or a pharmaceutically acceptable salt thereof, wherein A is

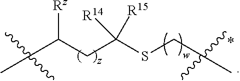

3. A compound of claim 2, or a pharmaceutically acceptable salt thereof, wherein:
z is 0;
w is 1; and
R^{z} is —C(O)NHR¹⁶.

4. A compound of claim 3, or a pharmaceutically acceptable salt thereof, wherein R¹⁶ is hydrogen or CHR¹⁷C(O)NH₂, wherein R¹⁷ is hydrogen.

5. A compound of any one of claims 1-4, or a pharmaceutically acceptable salt thereof, wherein:
R^{d} is methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;
R^{g} is methyl, or R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group; and
R^{k} is methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy.

6. A compound of any one of claims 1-4, or a pharmaceutically acceptable salt thereof, wherein:
R^{d} and R⁴, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;
R^{g} and R⁷, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group; and
R^{k} is methyl.

7. A compound of any one of claims 1-4, or a pharmaceutically acceptable salt thereof, wherein:
R¹ is biphenylC₁-C₃alkyl wherein the biphenyl is optionally substituted with a methyl group, diphenylmethyl, naphthylC₁-C₃alkyl, phenoxyC₁-C₃alkyl wherein the phenoxy part of the phenoxyC₁-C₃alkyl is optionally substituted with a C₁-C₃alkyl group, or phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, or —OP(O)(OH)₂;
R² is C₁-C₇alkyl, C₂-C₇alkenyl, C₁-C₃alkoxyC₁-C₃alkyl, or C₁-C₃alkylsulfanylC₁-C₃alkyl, or, R² and R^{b}, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxyl;
R³ is C₁-C₆alkoxycarbonylC₁-C₃alkyl, carboxyC₁-C₃alkyl, or NR^{t}R^{u}carbonylC₁-C₃alkyl, wherein R^{t} and R^{u} are independently hydrogen, C₁-C₃alkyl, or triphenylmethyl;
R⁴ and R^{d}, together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;
R⁵ is —(CH₂)_{q}NR⁵⁰R⁵¹;
R⁶ is C₁-C₇alkyl, C₂-C₇alkenyl, C₁-C₃alkoxyC₁-C₃alkyl, or C₁-C₃alkylsulfanylC₁-C₃alkyl;
R⁷ and R^{g}, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;
R⁸ and R¹⁰ are each independently azaindolylC₁-C₃alkyl, benzothiazolylC₁-C₃alkyl, benzothienylC₁-C₃alkyl, benzyloxyC₁-C₃alkyl, C₃-C₁₄cycloalkylC₁-C₃alkyl, furanylC₁-C₃alkyl, imidazolylC₁-C₃alkyl, pyridinylC₁-C₃alkyl, thiazolylC₁-C₃alkyl, thienylC₁-C₃alkyl, or indolylC₁-C₃alkyl, wherein the indolyl part of the indolylC₁-C₃alkyl is optionally substituted with one group which is C₁-C₆alkoxycarbonyl, C₁-C₆alkoxycarbonylC₁-C₃alkyl, C₁-C₃alkyl, carboxyC₁-C₃alkyl, halo, haloC₁-C₃alkoxycarbonyl, hydroxy, or phenyl, wherein the phenyl is further optionally substituted by one, two, or three groups wherein each group is independently C₁-C₃alkoxy, C₁-C₃alkyl, or halo;
R⁹ is —(CH₂)_{q}NR^{50′}R^{51′}; and
R¹¹, R¹², and R¹³ are each independently C₁-C₇alkyl, C₂-C₇alkenyl, C₁-C₃alkoxyC₁-C₃alkyl, or C₁-C₃alkylsulfanylC₁-C₃alkyl.

8. A compound of claim 7, or a pharmaceutically acceptable salt thereof, wherein:
R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;
R² is C₁-C₇alkyl, or, R² and R^{b}, together with the atoms to which they are attached, form piperidine ring;
R³ is NR^{t}R^{u}carbonylC₁-C₃alkyl, wherein R^{t} and R^{u} are independently hydrogen or C₁-C₃alkyl;
R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁵ is —(CH₂)_{q}NR⁵⁰R⁵¹;
R⁶ is C₁-C₇alkyl;
R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;
R⁹ is —(CH₂)_{q′}NR^{50′}R^{51′}; and
R¹¹, R¹², and R¹³ are each independently C₁-C₇alkyl.

9. A compound of any one of claims 1-4, or a pharmaceutically acceptable salt thereof, wherein:
R⁵ is —(CH₂)_{q}NR⁵⁰R⁵¹;
R⁹ is —(CH₂)_{q′}NR^{50′}R^{51′}; and
R⁵⁰, R⁵¹, R^{50′}, and R^{51′} are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹; provided that when R⁵⁰ and R⁵¹ are each hydrogen, at least one of R^{50′} and R^{51′} is other than hydrogen.

10. A compound of any one of claims 1-4, or a pharmaceutically acceptable salt thereof, wherein:
R⁵ is —(CH₂)_{q}NR⁵⁰R⁵¹;
R⁹ is —(CH₂)_{q′}NR^{50′}R^{51′}; and
R⁵⁰, R⁵¹, R^{50′}, and R^{51′} are each independently hydrogen, C₁-C₁₃alkoxycarbonyl, C₄-C₁₃alkylcarbonyl, or C₁-C₁₃haloalkylcarbonyl; provided that when R⁵⁰ and R⁵¹ are each hydrogen, at least one of R^{50′} and R^{51′} is other than hydrogen.

11. A compound of claim 1, or a pharmaceutically acceptable salt thereof, wherein:
A is 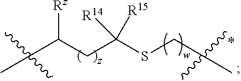
z is 0;
w is 1;
R^{z} is —C(O)NHR¹⁶;
R¹⁶ is hydrogen or CHR¹⁷C(O)NH₂, wherein R¹⁷ is hydrogen;
R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;
R² is C₁-C₇alkyl, or, R² and R^{b}, together with the atoms to which they are attached, form piperidine ring;
R³ is NR^{t}R^{u}carbonylC₁-C₃alkyl, wherein R^{t} and R^{u} are independently hydrogen or C₁-C₃alkyl;
R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁵ is —(CH₂)_{q}NR⁵⁰R⁵¹;
R⁶ is C₁-C₇alkyl;
R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;
R⁹ is —(CH₂)_{q′}NR^{50′}R^{51′}; and
R¹¹, R¹², and R¹³ are each independently C₁-C₇alkyl.

12. A compound of claim 1, or a pharmaceutically acceptable salt thereof, wherein:
A is 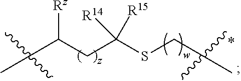
z is 0;
w is 1;
R^{z} is —C(O)NHR¹⁶;
R¹⁶ is hydrogen or CHR¹⁷C(O)NH₂, wherein R¹⁷ is hydrogen;
R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;
R² is C₁-C₇alkyl, or, R² and R^{b}, together with the atoms to which they are attached, form piperidine ring;
R³ is NR^{t}R^{u}carbonylC₁-C₃alkyl, wherein R^{t} and R^{u} are independently hydrogen or C₁-C₃alkyl;
R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁵ is —(CH₂)_{q}NR⁵⁰R⁵¹; wherein R⁵⁰ and R⁵¹ are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹;
R⁶ is C₁-C₇alkyl;
R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;
R⁹ is —(CH₂)_{q′}NR^{50′}R^{51′}; wherein R^{50′} and R^{51′} are each independently hydrogen, C₁-C₁₃alkylsulfanylcarbonyl, C₁-C₁₃haloalkoxycarbonyl, —CN, —C(N—CN)C₁-C₁₃alkyl, —C(O)NR⁷⁰R⁷¹, —C(S)NR⁹⁰R⁹¹, or —SO₂NR⁹⁰R⁹¹; provided that when R⁵⁰ and R⁵¹ are each hydrogen, at least one of R^{50′} and R^{51′} is other than hydrogen; and
R¹¹, R¹², and R¹³ are each independently C₁-C₇alkyl.

13. A compound of claim 1, or a pharmaceutically acceptable salt thereof, wherein:
A is 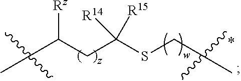
z is 0;
w is 1;
R^{z} is —C(O)NHR¹⁶;
R¹⁶ is CHR¹⁷C(O)NH₂, wherein R¹⁷ is hydrogen;
R¹ is phenylC₁-C₃alkyl wherein the phenyl part of the phenylC₁-C₃alkyl is optionally substituted with one, two, three, four, or five groups wherein each group is independently C₁-C₄alkoxy, C₁-C₄alkyl, amino, aminoC₁-C₃alkyl, carboxy, cyano, halo, haloC₁-C₃alkyl, hydroxy, or —OP(O)(OH)₂;
R² is C₁-C₇alkyl, or, R² and R^{b}, together with the atoms to which they are attached, form piperidine ring;
R³ is NR^{t}R^{u}carbonylC₁-C₃alkyl, wherein R^{t} and R^{u} are independently hydrogen or C₁-C₃alkyl;
R⁴ and R^{d}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁵ is —(CH₂)_{q}NR⁵⁰R⁵¹; wherein R⁵⁰ and R⁵¹ are each independently hydrogen, C₁-C₁₃alkoxycarbonyl, C₄-C₁₃alkylcarbonyl, or C₁-C₁₃haloalkylcarbonyl;
R⁶ is C₁-C₇alkyl;
R⁷ and R^{g}, together with the atoms to which they are attached, form a pyrrolidine, morpholine, piperidine, or piperazine ring, wherein each ring is optionally substituted with one to four groups wherein each group is independently amino, cyano, methyl, halo, or hydroxy;
R⁸ and R¹⁰ are each independently azaindolylC₁-C₃alkyl or indolylC₁-C₃alkyl, wherein the indolyl part of the indolylC₁-C₃alkyl is optionally substituted with one group which is C₁-C₃alkoxycarbonylC₁-C₃alkyl, C₁-C₃alkyl, carboxyC₁-C₃alkyl, halo, or hydroxy;
R⁹ is —(CH₂)_{q}NR^{50′}R^{51′}; wherein R^{50′} and R^{51′} are each independently hydrogen, C₁-C₁₃alkoxycarbonyl, C₄-C₁₃alkylcarbonyl, or C₁-C₁₃haloalkylcarbonyl; provided that when R⁵⁰ and R⁵¹ are each hydrogen, at least one of R^{50′} and R^{51′} is other than hydrogen; and
R¹¹, R¹², and R¹³ are each independently C₁-C₇alkyl.

14. A compound of formula (II) 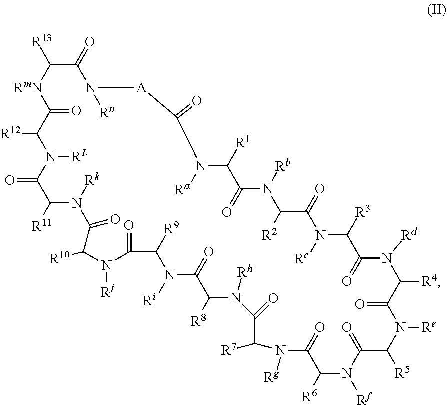
or a pharmaceutically acceptable salt thereof, wherein:
A is selected from a bond, 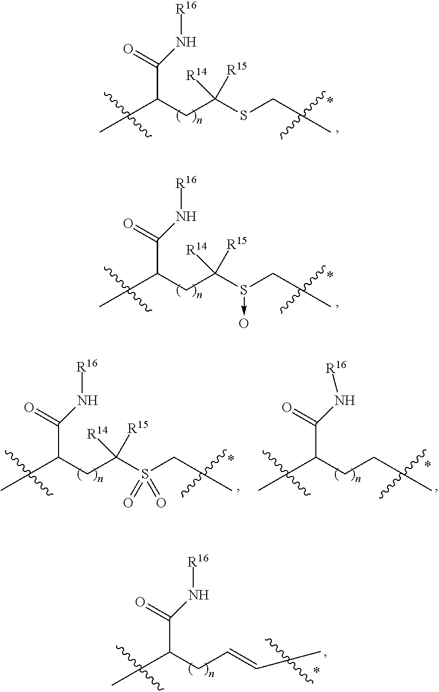
and;
wherein: [symbol]
denotes the point of attachment to the carbonyl group and [symbol] denotes the point of attachment to the nitrogen atom;
n is 0 or 1;
m is 1 or 2;
R¹⁴ and R¹⁵ are independently hydrogen or methyl; and
R¹⁶ is hydrogen, —CHR¹⁷C(O)NH₂, —CHR¹⁷C(O)NHCHR¹⁸C(O)NH₂, or —CHR¹⁷C(O)NHCHR¹⁸C(O)NHCH₂C(O)NH₂; wherein R¹⁷ is hydrogen or —CH₂OH and wherein R¹⁸ is hydrogen or methyl;
R^{c}, R^{f}, R^{h}, Rⁱ, and R^{m} are hydrogen;
Rⁿ is methyl;
R^{a} and R^{j}, are each independently hydrogen or methyl;
q and q′ are each independently 1 or 2;
R¹, R², R³, R⁴, R⁵, R⁶, R⁷, R⁸, R⁹, R¹⁰, R¹¹, R¹², and R¹³ are each independently a natural amino acid side chain or an unnatural amino acid side chain; or form a ring with the corresponding vicinal R group as described below;
R^{b} is methyl or R^{b} and R², together with the atoms to which they are attached, form an azetidine, pyrrolidine, morpholine, piperidine, piperazine, or tetrahydrothiazole ring; wherein each ring is optionally substituted with one to four groups wherein each group is independently amino, cyano, methyl, halo, or hydroxy;
R^{d} is hydrogen or methyl, or R^{d} and R⁴, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, hydroxy, or phenyl;
R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy;
R^{e} is hydrogen or methyl, or R^{e} and R⁵, together with the atoms to which they are attached, can form an azetidine, pyrrolidine, morpholine, piperidine, piperazine, or tetrahydrothiazole ring; wherein each ring is optionally substituted with one to four groups wherein each group is independently amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, or tetrazolyl; and wherein the pyrrolidine ring and the piperidine ring are optionally fused to a cyclohexyl, phenyl, or indole group;
R^{k} is hydrogen or methyl, or R^{k} and R¹¹, together with the atoms to which they are attached, can form an azetidine, pyrrolidine, morpholine, piperidine, piperazine, or tetrahydrothiazole ring; wherein each ring is optionally substituted with one to four groups wherein each group is independently amino, cyano, methyl, halo, and hydroxy;
R^{L} is methyl or R^{L} and R¹², together with the atoms to which they are attached, form an azetidine or pyrrolidine ring, wherein each ring is optionally substituted with one to four groups wherein each group is independently amino, cyano, methyl, halo, or hydroxy;
provided that the compound of formula (I) contains at least one carbon on the backbone of the ring that has four substituents other than hydrogen and is not an alpha-methyl-substituted ring.

15. A method of enhancing, stimulating, and/or increasing an immune response in a subject in need thereof, wherein the method comprises administering to the subject a therapeutically effective amount of a compound of any one of claims 1-14, or a pharmaceutically acceptable salt thereof.

16. A method of blocking the interaction of PD-L1 with PD-1 and/or CD80 in a subject, wherein the method comprises administering to the subject a therapeutically effective amount of a compound of any one of claims 1-14 or a pharmaceutically acceptable salt thereof.


SEQUENCE LISTING (2 sequences)

SEQ ID NO: 1 - protein, 384 aa, Homo sapiens
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGSPGGGGGREPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Homo sapiens
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSSETVRFQGHHHHHH